(19) United States
(12) Patent Application Publication
(10) Pub. No.: US 2023/0255564 A1
(43) Pub. Date: Aug. 17, 2023

(54) SYSTEMS AND METHODS FOR MACHINE-LEARNING-ASSISTED COGNITIVE EVALUATION AND TREATMENT

(71) Applicant: Linus Health, Inc., Boston, MA (US)

(72) Inventors: Alvaro Pascual-Leone, Boston, MA (US); William Souillard-Mandar, Boston, MA (US); Emily Rogers, Boston, MA (US); Jeff Bacon, Boston, MA (US); John Langton, Boston, MA (US); Sean Tobyne, Boston, MA (US); Karl Thompson, Boston, MA (US); David Bates, Boston, MA (US)

(21) Appl. No.: 18/125,467

(22) Filed: Mar. 23, 2023

Related U.S. Application Data
(63) Continuation of application No. PCT/US21/52218, filed on Sep. 27, 2021.
(60) Provisional application No. 63/083,266, filed on Sep. 25, 2020.

(51) Int. Cl. A61B 5/00; A61B 5/0205; G16H 50/20

(52) U.S. Cl. CPC A61B 5/7264; A61B 5/0205; G16H 50/20; A61B 5/16

(57) ABSTRACT

Systems, methods, and computer program products are provided for determining one or more biomarker and/or health condition of a target patient. In various embodiments, a method is provided where a plurality of health data of the target patient and/or a plurality of first order features determined from the plurality of health data of the target patient are received as input to a pre-trained artificial neural network. The plurality of health data is derived from a plurality of modalities. A plurality of latent variables based on the plurality of health data and plurality of first order features are received from an intermediate layer of the pre-trained artificial neural network. The plurality of latent variables are provided to a pre-trained learning system. The pre-trained learning system is trained to receive as input the plurality of latent variables and output one or more biomarker and/or health condition of the target patient.

### CROSS-REFERENCE TO RELATED APPLICATION

This application is a continuation of PCT Application No. PCT/US2021/52218, filed Sep. 27, 2021, which claims the benefit of U.S. Provisional Application No. 63/083,266, filed Sep. 25, 2020, both of which are hereby incorporated by reference in their entirety.

### TECHNICAL FIELD

Embodiments of the disclosure generally relate to the field of determining biomarkers and/or health conditions of patients from multimodal health data via machine learning.

### BACKGROUND

Cognitive impairment, specifically dementia and Alzheimer's disease, is one of the largest health problems in the United States. There are approximately 6 million individuals in the U.S. with some form of dementia, representing an annual cost to the healthcare system of 225 billion. Approximately 5.3 million of these people have Alzheimer's disease, the 6th leading cause of death in the U.S. By 2050, these numbers are expected to almost triple to nearly 16 million Americans diagnosed with dementia, with an annual cost of more than 1 trillion. Current standards of care to address this enormous health problem are often lengthy for both practitioners and patients, potentially invasive, expensive, and may not detect impairment early enough to intervene and potentially change the course of disease. There is a need for cost effective, reliable, objective, noninvasive, accurate, systems to identify and track meaningful deviations in brain health and to detect cognitive impairment at its earliest stages. In addition, there is a growing need to optimize care and treatment recommendation, as well as dosage and personalization of existing and in-development therapies.

Accordingly, there is a need for improved methods and systems for determining biomarkers and/or health conditions of a patient related to cognitive health from multimodal data relating to the patient.

### BRIEF SUMMARY

In various embodiments, a method of determining one or more biomarker and/or health condition of a target patient is provided where a plurality of health data of the target patient and/or a plurality of first order features determined from the plurality of health data of the target patient are received as input to a pre-trained artificial neural network. The plurality of health data is derived from a plurality of modalities. A plurality of latent variables based on the plurality of health data and plurality of first order features are received from an intermediate layer of the pre-trained artificial neural network. The plurality of latent variables are provided to a pre-trained learning system. The pre-trained learning system is trained to receive as input the plurality of latent variables and output one or more biomarker and/or health condition of the target patient.

In various embodiments, a method of generating a digital model of a target patient is provided where a plurality of health data of the target patient and/or a plurality of first order features determined from the plurality of health data of the target patient is received as input to an artificial neural network. The plurality of health data of the target patient is derived from a plurality of modalities. The artificial neural network is trained to generate, at an intermediate layer thereof, a plurality of latent variables based on the plurality of health data and/or plurality of first order features of the target patient.

In various embodiments, a method is provided of training a system to determine one or more biomarker and/or health condition of a target patient where a plurality of health data and/or a plurality of first order features determined from the plurality of health data is received as input to a first artificial neural network. The plurality of health data is derived from a plurality of modalities. The first artificial neural network is trained to generate, at an intermediate layer thereof, a plurality of latent variables based on the plurality of health data and/or plurality of first order features. A second artificial neural network is trained to output one or more biomarker and/or health condition based on the plurality of latent variables.

In various embodiments, a method of synthesizing health data of a target patient is provided where a plurality of health data of the target patient and/or a plurality of first order features determined from the plurality of health data of the target patient is received as input to a pre-trained artificial neural network. The plurality of health data of the target patient is derived from a plurality of modalities. A plurality of latent variables based on the plurality of health data and/or plurality of first order features of the target patient are received from an intermediate layer of the pre-trained artificial neural network. The plurality of latent variables are provided to a pre-trained learning system. The plurality of health data and/or the plurality of first order features are provided to the pre-trained learning system. The pre-trained learning system is trained to receive as input the plurality of latent variables and at least one of the plurality of health data and/or the first order features. The pre-trained learning system is configured to synthesize at least one value associated with the plurality of health data and/or the first order features.

In various embodiments, a system for determining one or more biomarker and/or health condition of a target patient is provided. The system includes a computing node with a computer readable storage medium having program instructions embodied therewith. The program instructions are executable by a processor of the computing node to cause the processor to perform a method where a plurality of health data of the target patient and/or a plurality of first order features determined from the plurality of health data of the target patient are received as input to a pre-trained artificial neural network. The plurality of health data is derived from a plurality of modalities. A plurality of latent variables based on the plurality of health data and plurality of first order features are received from an intermediate layer of the pre-trained artificial neural network. The plurality of latent variables are provided to a pre-trained learning system. The pre-trained learning system is trained to receive as input the plurality of latent variables and output one or more biomarker and/or health condition of the target patient.

In various embodiments, a system for generating a digital model of a target patient is provided. The system includes a computing node with a computer readable storage medium having program instructions embodied therewith. The program instructions are executable by a processor of the computing node to cause the processor to perform a method where a plurality of health data of the target patient and/or a plurality of first order features determined from the plurality of health data of the target patient is received as input to an artificial neural network. The plurality of health data of the target patient is derived from a plurality of modalities. The artificial neural network is trained to generate, at an intermediate layer thereof, a plurality of latent variables based on the plurality of health data and/or plurality of first order features of the target patient.

In various embodiments, a system for training a system to determine one or more biomarker and/or health condition of a target patient is provided. The system includes a computing node with a computer readable storage medium having program instructions embodied therewith. The program instructions are executable by a processor of the computing node to cause the processor to perform a method where a plurality of health data and/or a plurality of first order features determined from the plurality of health data is received as input to a first artificial neural network. The plurality of health data is derived from a plurality of modalities. The first artificial neural network is trained to generate, at an intermediate layer thereof, a plurality of latent variables based on the plurality of health data and/or plurality of first order features. A second artificial neural network is trained to output one or more biomarker and/or health condition based on the plurality of latent variables.

In various embodiments, a system for synthesizing health data of a target patient is provided. The system includes a computing node with a computer readable storage medium having program instructions embodied therewith. The program instructions are executable by a processor of the computing node to cause the processor to perform a method where a plurality of health data of the target patient and/or a plurality of first order features determined from the plurality of health data of the target patient is received as input to a pre-trained artificial neural network. The plurality of health data of the target patient is derived from a plurality of modalities. A plurality of latent variables based on the plurality of health data and/or plurality of first order features of the target patient are received from an intermediate layer of the pre-trained artificial neural network. The plurality of latent variables are provided to a pre-trained learning system. The plurality of health data and/or the plurality of first order features are provided to the pre-trained learning system. The pre-trained learning system is trained to receive as input the plurality of latent variables and at least one of the plurality of health data and/or the first order features. The pre-trained learning system is configured to synthesize at least one value associated with the plurality of health data and/or the first order features.

In various embodiments, a computer program product for determining one or more biomarker and/or health condition of a target patient is provided. The computer program product includes a computer readable storage medium having program instructions embodied therewith. The program instructions are executable by a processor to cause the processor to perform a method where a plurality of health data of the target patient and/or a plurality of first order features determined from the plurality of health data of the target patient are received as input to a pre-trained artificial neural network. The plurality of health data is derived from a plurality of modalities. A plurality of latent variables based on the plurality of health data and plurality of first order features are received from an intermediate layer of the pre-trained artificial neural network. The plurality of latent variables are provided to a pre-trained learning system. The pre-trained learning system is trained to receive as input the plurality of latent variables and output one or more biomarker and/or health condition of the target patient.

In various embodiments, a computer program product for generating a digital model of a target patient is provided. The computer program product includes a computer readable storage medium having program instructions embodied therewith. The program instructions are executable by a processor to cause the processor to perform a method where a plurality of health data of the target patient and/or a plurality of first order features determined from the plurality of health data of the target patient is received as input to an artificial neural network. The plurality of health data of the target patient is derived from a plurality of modalities. The artificial neural network is trained to generate, at an intermediate layer thereof, a plurality of latent variables based on the plurality of health data and/or plurality of first order features of the target patient.

In various embodiments, a computer program product f for training a system to determine one or more biomarker and/or health condition of a target patient is provided. The computer program product includes a computer readable storage medium having program instructions embodied therewith. The program instructions are executable by a processor to cause the processor to perform a method where a plurality of health data and/or a plurality of first order features determined from the plurality of health data is received as input to a first artificial neural network. The plurality of health data is derived from a plurality of modalities. The first artificial neural network is trained to generate, at an intermediate layer thereof, a plurality of latent variables based on the plurality of health data and/or plurality of first order features. A second artificial neural network is trained to output one or more biomarker and/or health condition based on the plurality of latent variables.

In various embodiments, a computer program product for synthesizing health data of a target patient is provided. The computer program product includes a computer readable storage medium having program instructions embodied therewith. The program instructions are executable by a processor to cause the processor to perform a method where a plurality of health data of the target patient and/or a plurality of first order features determined from the plurality of health data of the target patient is received as input to a pre-trained artificial neural network. The plurality of health data of the target patient is derived from a plurality of modalities. A plurality of latent variables based on the plurality of health data and/or plurality of first order features of the target patient are received from an intermediate layer of the pre-trained artificial neural network. The plurality of latent variables are provided to a pre-trained learning system. The plurality of health data and/or the plurality of first order features are provided to the pre-trained learning system. The pre-trained learning system is trained to receive as input the plurality of latent variables and at least one of the plurality of health data and/or the first order features. The pre-trained learning system is configured to synthesize at least one value associated with the plurality of health data and/or the first order features.

### BRIEF DESCRIPTION OF THE DRAWINGS

FIG. 1 illustrates a system diagram showing information flow according to embodiments of the present disclosure.

FIG. 2 illustrates a flowchart illustrating a patient experience process flow according to embodiments of the present disclosure.

FIG. 3 illustrates a system diagram showing information flow in an embodiment focused on two tasks to collect first-order features according to embodiments of the present disclosure.

FIG. 4 illustrates a notional representation of time series data collected from multiple different sources (i.e. multimodal) to be used in further analysis according to embodiments of the present disclosure.

FIGS. 5A-5B illustrate an exemplary neural network for predicting a MOCA score from multimodal data according to embodiments of the present disclosure.

FIG. 6 illustrates a method of calculating a time-windowed aggregation according to embodiments of the present disclosure.

FIGS. 7A-7B illustrate a machine learning workflow for synthesizing missing data points of health data within a time series according to embodiments of the present disclosure.

FIGS. 8A-8B illustrates an exemplary clustering of disease codes according to embodiments of the present disclosure.

FIGS. 9A-9B illustrate a machine learning workflow for synthesizing missing health data in a modality from a plurality of other modalities according to embodiments of the present disclosure.

FIG. 10 illustrates a Deep-Q learning workflow for optimizing intervention recommendations according to embodiments of the present disclosure.

FIG. 11 illustrates a workflow showing a feedback loop of determining clinical recommendations based on patient health data for clinician review according to embodiments of the present disclosure.

FIG. 12 illustrates an exemplary workflow of a patient data model (a “digital twin”) according to embodiments of the present disclosure.

FIG. 13 illustrates an exemplary model leveraging first and second order features to predict the onset of Alzheimer's disease according to embodiments of the present disclosure.

FIG. 14 illustrates an exemplary feature grouping and determination of importance for per-patient features according to embodiments of the present disclosure.

FIG. 15 depicts an exemplary computing node according to embodiments of the present disclosure.

### DETAILED DESCRIPTION

In various embodiments, the present disclosure provides systems, methods, and computer program products for machine-learning-assisted determination of patient biomarkers and/or health conditions, and generation of a latent representation of patient cognitive health. In various embodiments, a system can administer a series of cognitive assessments to an individual to capture raw health data regarding the patient (e.g., speech, gait and balance, eye motion, drawing, sleep, facial expressions, gestures) from various different modalities, generate first-order features from the raw health data derived from these data, and relate those to specific brain health domains, clinical diagnoses, and/or treatment plans.

In various embodiments, the present disclosure may integrate data received from health tasks across multiple modalities captured using smartphone, tablet, or other sensors to generate aggregate measures of brain function for different cognitive biomarkers and/or diagnoses. In various embodiments, health data from the various modalities may be provided to a machine learning system to thereby generate associations between the data. In various embodiments, the associations may be generated in the form of latent variables extracted from the machine learning system (e.g., a neural network). In various embodiments, the latent variables may be extracted from an intermediate layer of the neural network. In various embodiments, recommendations for optimized care and treatment actions may be provided based on these associations. In various embodiments, a platform may be optimized to be more sensitive to cognitive decline and more specific to particular neurological diseases than existing individual end-point solutions by themselves via the determined associations between various data modalities and within individual data sets. In various embodiments, the platform can select tasks across various complementary neurological systems that have been shown in published research to be correlated with brain health and different brain domains. In various embodiments, the tasks and/or assessments may include: drawing-based tasks, measures of decision making and reaction time, speech elicitation tasks, eye tracking-based memory assessments, gait and balance assessments, sleep measurements, and a lifestyle/health history questionnaire.

In various embodiments, first-order features of brain health may be extracted from these data. In various embodiments, first order features may include any transformation of raw recorded health data, or insights derived from clinical expertise that may not be explicitly quantified. In various embodiments, the first-order features and the raw health data may be input to a machine learning algorithm (e.g., a recurrent neural network) trained on subject brain health information (e.g., neuropsychological test scores, blood and brain imaging biomarkers, clinical consensus diagnoses, etc.) to generate second-order features tied to specific brain health domains (e.g., memory, motor control, executive function), specific brain areas and networks (e.g., right or left hippocampal formation, right or left prefrontal cortex, right or left attentional network), and clinical diagnoses (e.g., Alzheimer's Disease, Parkinson's Disease). In various embodiments, the second-order features may be latent variables extracted from an intermediate layer of the neural network. In various embodiments, the second-order features may be provided to other pre-trained machine learning algorithms trained for other tasks, such as predicting a MoCA score, synthesizing likely EEG data, synthesizing a likely fMRI image, identifying affected brain regions, pathways, or circuits, and optimize care and treatment recommendation, as well as dosage and personalization of existing and in-development therapies.

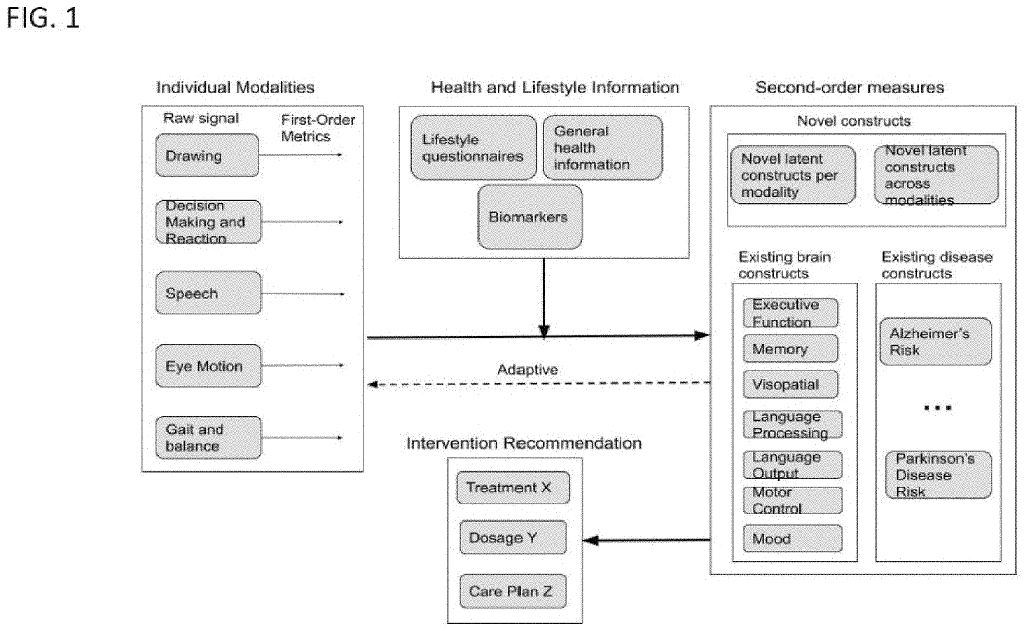

FIG. 1 shows the manner in which information flows in some embodiments in accordance with the present disclosure. In various embodiments, a system may collect health data from tasks and/or assessments provided to the patient using suitable hardware (e.g., tablet, smartphone). In various embodiments, exemplary assessments include drawing assessments, decision making and reaction assessments, speech assessments, eye motion assessments, gait and balance assessments, and/or sleep assessments. In various embodiments, the collected information may then be encrypted and securely stored in a database associated with the platform. In various embodiments, based on the recorded data, the system may determine first-order features as described in more detail herein.

In various embodiments, the first-order features and raw health data may be provided to a machine learning system to generate second-order features. In various embodiments, the second-order features may include novel constructs (e.g., novel latent constructs per modality and/or across multiple modalities), existing brain constructs (e.g., memory, executive function), and associated disease constructs (e.g., potential for Alzheimer's disease, Parkinson's disease, frontotemporal dementia). In various embodiments, existing brain constructs may also include affected regions (e.g., mesial temporal lobe, Broca's area) and neural circuits (e.g., Papez circuit) or systems (e.g., limbic system).

In various embodiments, after or during an initial collection of health data, the system may prompt further or additional collection based on an adaptive task administration. For example, for a given set of individual tasks captured and second-order features derived from them, the system may prompt the patient, physician, or patient care team to capture more individual tasks and repeat the process of generating the second-order features to generate updated second-order features.

In various embodiments, the system may provide the second-order features to a pre-trained machine learning system trained to perform a specific task based on the provided second order features, such as providing recommendations and/or diagnoses. In various embodiments, the system can identify one or more abnormal constructs, regions, circuits, or pathways in the brain. In various embodiments, the system may recommend specific treatments or confirmatory testing that the patient is not able to have done without elements beyond the system (e.g., MRI, CT scan). In various embodiments, based on the patient data and calculated second-order features, the system may personalize the recommended treatment by, for example, personalizing a dosage or recommending follow up visits to a particular professional or clinic accessible to the patient (e.g., referral to neurologist vs. psychiatrist).

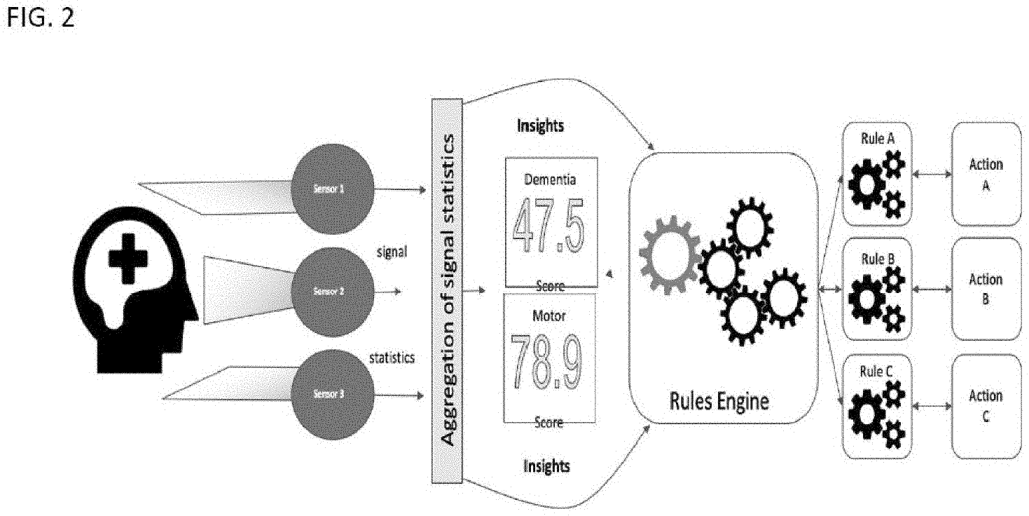

FIG. 2 illustrates a patient experience process flow. In various embodiments, a series of tasks may be administered to an individual. In various embodiments, administration may involve using a personal computer, laptop, tablet, smartphone, smartwatch, activity tracker, or the like, to ease deployment by leveraging equipment more commonly found in clinical settings and that require less maintenance, and may result in a concomitant decrease of cost and administrator burden. In various embodiments, task data captured by the device(s) can be securely transmitted to the system's servers where it can be decrypted, then analyzed using advanced analytics. In various embodiments, following test analysis, a report may be automatically generated and may provide for immediate availability for review by, for example, clinical staff, administrators, or the patients themselves. In various embodiments, the results and recommendations generated by the analysis of the tasks may then be used clinically for a more accurate assessment of cognitive function and brain health.

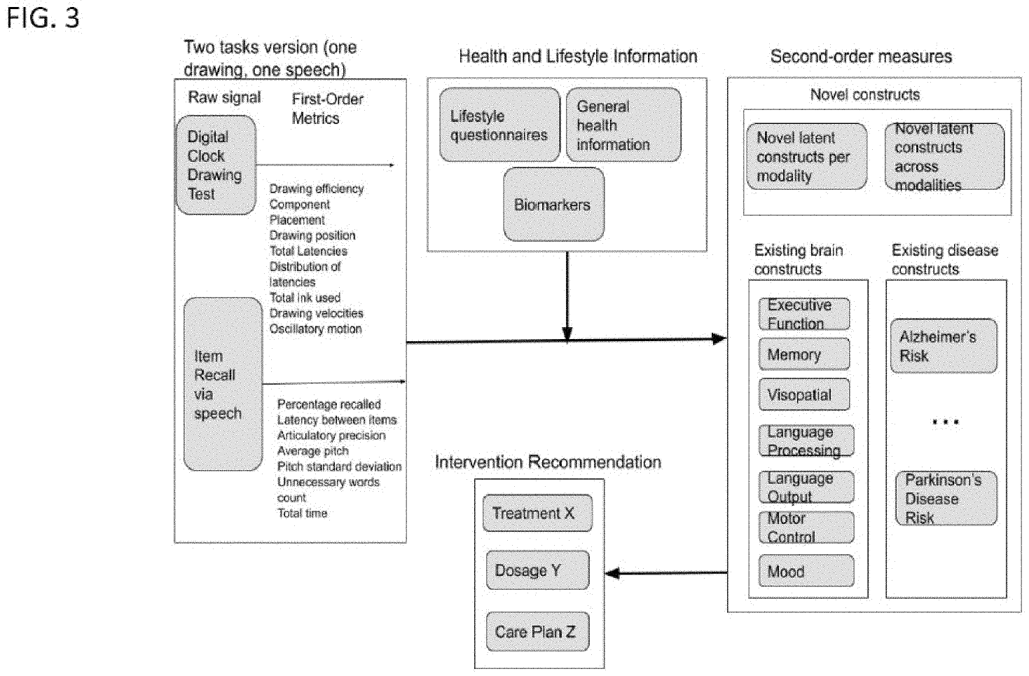

In various embodiments, the information flow of an exemplary system in accordance with the present disclosure that administers two tasks to collect first-order features is depicted in FIG. 3. In this example, the system may begin by prompting a patient to complete two tasks: a clock drawing task and an item recall speech task. The behavior signal elicited from the tasks can be measured to collect modalities and first-order features. For example, a clock drawing task may allow the system to measure elements such as: drawing efficiency, correct component placement, drawing position, distribution of latencies, total ink used, drawing velocities, and oscillatory motion. An item recall speech test may allow the system to measure elements such as: percentage recalled, latency between items, hesitations, articulatory precision, average pitch, and unnecessary words count. In various embodiments, these first-order features may be provided with the raw data to a machine learning system used to generate second-order features, for example, a novel latent construct based on the combination of executive function measures from both the digital clock drawing task and the item recall task. In various embodiments, these second-order features may also be related to cognitive health measures including executive function, visuospatial reasoning, and memory. In various embodiments, the system may analyze the second-order features to assess existing disease constructs such as risk of Alzheimer's or Parkinson's disease. In various embodiments, an intervention recommendation, that may include the identification of relevant risks (e.g., high risk for postoperative delirium), recommendations for specific treatment (e.g., avoid specific anesthesia medicines), or the suggestion of a particular care plan, can then be communicated to a patient, physician, or patient care team.

In various embodiments, similar metrics that measure complementary aspects of physical, neurological, and/or psychological health may be combined into a reduced set of features that captures relevant information. For example, the second order features of memory can be tested by various metrics, including, for example, immediate and delayed story recall, object recall, pattern recognition/matching, and execution time of verbal instruction. In various embodiments, various unsupervised or self-supervised methods can be used to extract a condensed second order features of memory. In various embodiments, dimensionality reduction methods can include, for example: removing metric correlation with principal component analysis (PCA, possibly in truncated form), visualizing similarities and differences with t-Distributed Stochastic Neighbor Embedding (t-SNE) or Uniform Manifold Approximation and Projection (UMAP), or learning non-linear relations with a deep learning autoencoder (AE). In various embodiments, these exemplary methods may perform dimensionality reduction, and provide a more compact latent space representation that can be used as components to the second order features of memory.

In various embodiments, additional processing may be done on the latent space representation by performing unsupervised clustering, such as density-based spatial clustering of applications with noise (DBSCAN), spectral clustering, and/or a hierarchical clustering using intrinsic optimality metrics like the silhouette score. In various embodiments, the formation of clusters can then be used to assign discrete classification scores to data, thus changing the second order features from multiple real valued components to single discrete classes. In various embodiments, future data may be processed similarly, either by rerunning classification on the transformed latent space representation, or by employing more static clustering methods like k-nearest neighbors (KNN) if time/processor constraints are present or if past clustering should not be changed. In various embodiments, by performing this kind of analysis on subcategories of metrics, higher order features of memory, executive function, fine and gross motor control, language processing, cognitive efficiency, spatial processing, information processing, psychological health, can be generated that may lend themselves to further combination or analysis.

In various embodiments, data structure can be learned in a supervised manner through clinical label such as diagnoses, neuropsychological testing scores, blood and brain biomarkers (e.g., amyloid, tau PET), and genetic risk factors (e.g., APoE). In various embodiments, various labels such as Alzheimer's, Parkinson's, progressive supranuclear palsy (PCP), mild cognitive impairment (MCI), pathological aging, or normal control can be assigned to samples through clinical diagnosis. In various embodiments, machine learning models such as linear regression, deep learning, random forests, and gradient boosters may be used to produce a prediction model for that clinical label, taking in either the raw data, or the computed second-order metrics which may allow faster processing and improved interpretability.

In various embodiments, the system may combine second-order features with specific medical information obtained from medical records or the user (e.g., blood and imaging biomarkers, genetic markers, standard neuropsychological tests) or more general health-related information (e.g., body mass index, medications, nutritional habits, frailty index, etc.). In various embodiments, the system may combine these features to gain additional insights as to the role different physical, neurological, and psychological subsystems play in contributing to changes in brain health and disease development.

In various embodiments, by assessing different components of health in these second-order features, the system may provide not only sensitivity to abnormal conditions, but also specificity to the exact nature of physical, cognitive or psychological decline.

In various embodiments, multimodal data may be input to the learning system (e.g., a neural network) used to determine a latent representation of a patient's health data for use in another learning system to determine a biomarker (e.g., a cognitive score) and/or health condition of the patient (e.g., cognitive disease). In various embodiments, the learning system can ingest patient data from health assessments administered via mobile devices. In various embodiments, the learning system can ingest patient data from integrated hardware devices (e.g., smart devices, fitness trackers, etc.), electronic health records (EHR) systems. In various embodiments, the learning system can ingest patient data from a third-party hardware, software, and/or service. In various embodiments, the learning system can ingest patient data from any suitable data sources such as: output from diagnostic tests administered via a mobile application as a part of the platform operating the learning system; output from diagnostic tests administered by third-party diagnostic devices and/or applications then ingested by a platform operating the learning system; output from integrated hardware devices (e.g., smart device, fitness tracker, etc.), connected home appliances, and/or general internet-of-things (IOT) devices. In various embodiments, the connected home appliances and/or IOT devices may be configured to record data regarding a user and/or the user's environment (e.g., frequency of use of an appliance/device, humidity, air quality, UV exposure, indoor/outdoor temperature, etc.); patient health data from electronic health records systems; patient health data obtained through clinicians to provide inputs and feedback pertaining to patient health and/or patient reported outcomes from surveys and/or ecological momentary assessments.

In various embodiments, multimodal data inputs may include: recording positional data of user interactions such as inputs (i.e., time stamped X-axis and Y-axis coordinates on a touch screen) provided by a mobile device stylus while the patient performs a task or assessment on a mobile application, such as drawing a clock; eye tracking data while providing visual stimulus and requiring the patient to perform tasks that elicit their ability to perceive and respond to that stimulus; audio recording while providing audiovisual stimulus and requiring the patient to vocalize responses to that stimulus to elicit their ability to perceive and respond to such stimulus; video data recording of patients performing some task such as walking; accelerometer data recording of patients performing some task such as walking; functional neuroimaging or sensing (e.g., electroencephalography (EEG) recordings or functional magnetic resonance imaging (fMRI) of the patient performing any one of the assessments or tasks listed herein; neuroimaging data from metabolic or chemical sources (e.g., positron emission tomography), structural or vascular imaging; data captured by third-party devices either contemporaneously with user performance of an assessment, or historically captured during day to day activity. For example, data from wearable personal health devices that can record pulse, galvanic responses, etc. may be provided as input to the learning system.

In various embodiments, the patient health data input to the learning system may include temporal data. In various embodiments, health data may be associated with timestamps of when each data point is captured and therefore has an element of temporality. In various embodiments, the interpretation of certain data inputs is particularly relative to analysis of the sequence of events over time. In various embodiments, temporal data inputs may generally refer to data points for a variable recorded over time, e.g., a time series of blood pressure over the course of a day. Examples of temporal data inputs include but are not limited to: time-stamped X-axis, Y-axis coordinates captured during a health assessment where the patient is asked to draw on a mobile device (an array of coordinates over time itself may be treated as a time series); coordinates captured over time related to tracking a patient's eyes while performing a health assessment that entails visual stimulus; audio signals captured over time as the patient responds to audiovisual stimulus from performing a health assessment; pulse data captured over the duration of a patient taking an assessment; EEG data captured over a predetermined period and/or while a patient performs a task or assessment; fMRI images captured over a predetermined period and/or while a patient performs a task or assessment.

In various embodiments, a notional example of temporal data captured while the subject is performing a health assessment such as clock drawing is shown in Table 1. In various embodiments, the timestamp includes hours, minutes, seconds, and samples per second. In various embodiments, samples per second may start indexing at 0 and max out based on the hardware device constraints. For example, 240 samples per second would mean the last 3 digits of the timestamp would never be greater than 239. In this example, once the samples per second value reached 239 it would be reset to 0 and the second would increment. In various embodiments, the sampling rate determines the resolution of data. In this example, with each sample (taken 240 times per second) we capture the X-coordinate, Y-coordinate, Azimuth, Altitude, and Force. In various embodiments, the ranges of these values may be determined by the hardware device specifications.

TABLE 1
Notional example of data captured during Linus
Health Assessment involving a drawing task.

| Timestamp | X | Y | Azimuth | Altitude | Force |
|---|---|---|---|---|---|
| 0200:21:02:001 | 253 | 453 | (.7, −.7) | 78° | 1 |
| 0200:21:02:002 | 254 | 452 | (.7, −.7) | 77° | 2 |
| 0200:21:02:003 | 255 | 451 | (.7, −.7) | 78° | 1 |

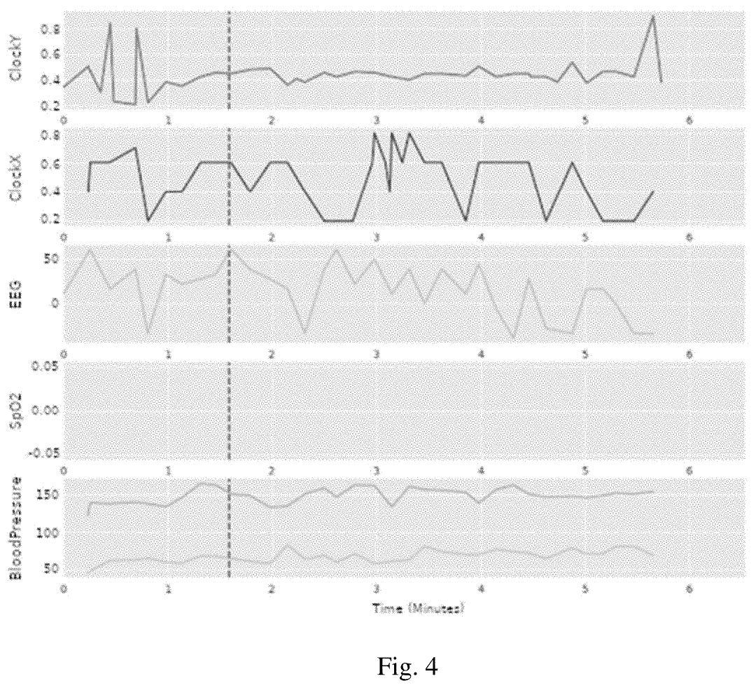

FIG. 4 illustrates a notional representation of time series data collected from multiple different sources (i.e. multimodal) to be used in further analysis. In various embodiments, multimodal, temporal data streams may be used in multivariate analysis and/or artificial intelligence applications.

In various embodiments, the raw data may include non-temporal data inputs. In various embodiments, non-temporal data inputs may include a temporal aspect related to when the data was captured. In various embodiments, interpretation of these inputs is generally less sensitive to the information of when they were collected or how they may change over time. Examples of non-temporal data inputs include but are not limited to: the blood type of a patient; genetic phenotyping of a patient; patient handedness (whether a patient is right or left handed); patient allergies or lack of allergies, and/or dietary and/or exercise habits.

In various embodiments, the raw data may be processed to determine features used to analyze the data in a machine learning system. In various embodiments, feature engineering may refer to both the use of raw data (e.g., recorded variables) and the construction of new variables from these raw data sources. In various embodiments, both raw data and constructed features are used as inputs to artificial intelligence algorithms. In various embodiments, feature engineering practices may differ for temporal versus non-temporal data. In various embodiments, features for artificial intelligence algorithms may include: features extracted with either no or minor transformation from raw data inputs; first order features derived from raw data inputs such as aggregations; second order features defined from subject matter expertise as aggregates of 1st order metrics and raw data, or features derived with algorithms or statistical methods executed on any of the raw data, first-order features, and/or the output of machine learning algorithms In various embodiments, first order features may include aggregations may be the results of statistics, machine learning algorithms, or rules generated from human subject matter expertise. In various embodiments, second order features may be determined by a machine learning system based on raw data and first-order features.

In various embodiments, some applications of machine learning systems may use relatively raw data inputs with minimal to no data transformation. In various embodiments, Recurrent Neural Networks (RNN) are one such application, where a general time window and sampling rate is used to input temporal data for training. An example of such a network being trained on clock drawing assessment data from Table 1 is shown in FIG. 4. In this case, the raw data are the X, Y coordinates, azimuth, altitude, and force inputs from direct user input during the clock drawing assessment. In various embodiments, there may be no transformation or feature engineering of the data. In various embodiments, a neural network (e.g., RNN) may learn latent features within hidden layers when trained on raw data. In various embodiments, time window subsets of the data may be provided during training of the network. As shown in FIG. 4, the time window includes only three samples from the notional data provided in Table 1. In various embodiments, the exact length of the input layer can be optimized using grid search techniques for different model architectures.

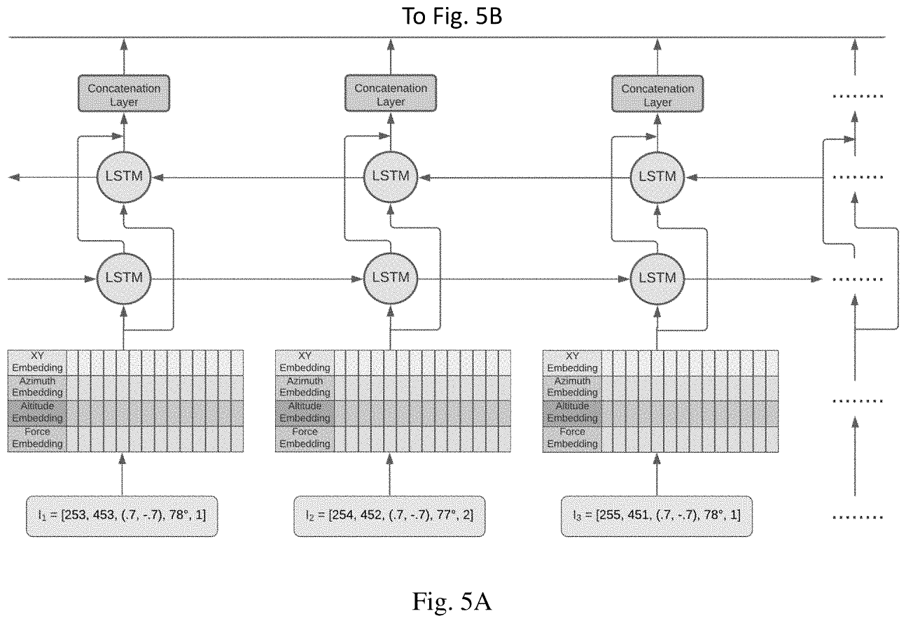

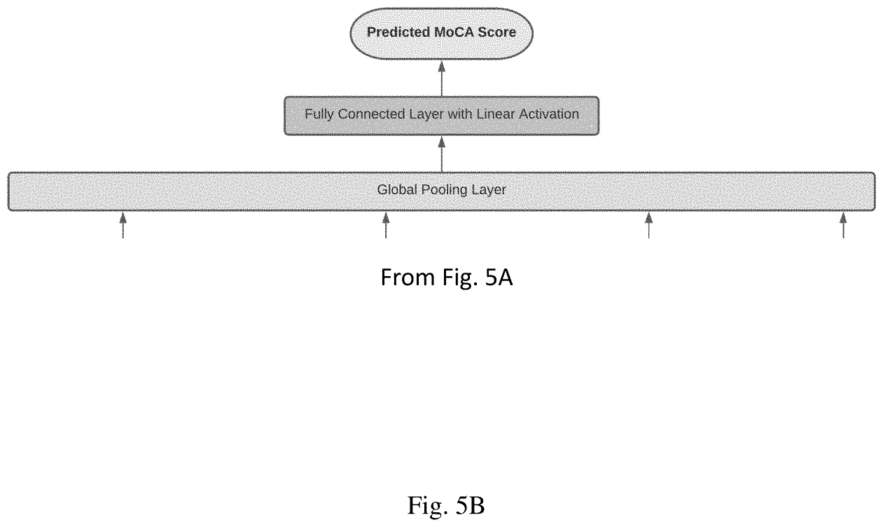

FIGS. 5A-5B illustrate an exemplary neural network for predicting a MOCA score from multimodal data. In particular, FIGS. 5A-5B illustrate an example application of a Long Short Term Memory version of an RNN to train on predicting a target variable of MOCA score. In various embodiments, the activation function may be selected to train a regression model. In various embodiments, the bidirectional nature of the LSTM may learn sequential associations between inputs that are predictive of MOCA score. In various embodiments, the LSTM may learn associations without the need for transforming the raw data inputs. In various embodiments, these approaches may generate latent features in the hidden layers of the network architecture which may be used to predicting the target variable. In various embodiments, values from the hidden layers or embeddings may themselves be used as first and/or second order features.

In various embodiments, as shown in the exemplary LSTM RNN exemplary model structure of FIGS. 5A-5B, data from a digital clock drawing assessment may be used as input. In various embodiments, embeddings for metrics from individual time points (bottom row), represented here as X coordinate, Y coordinate, Azimuth pair, Altitude, and Force, are learned in the first layer of the model (second row from bottom). In various embodiments, these embeddings are passed through the LSTM layers before concatenation and global pooling occurs. In various embodiments, the LSTM layers learn the sequence(s) of data. In various embodiments, a fully-connected layer with linear activation function produces a predicted MoCA score. In various embodiments, the fully connected layer may include one or more fully-connected layers. In various embodiments, the fully-connected layer(s) and linear activation function(s) may be replaced with any other suitable fully-connected layer(s) with linear activation function(s). In various embodiments, the fully-connected layer may be separately trained to output a particular result (e.g., MoCA scores). For example, the fully-connected layer with linear activation function layer to predict a MoCA score may be replaced with a fully-connected layer with linear activation function that predicts a result of another assessment, such as a speech test. In various embodiments, the fully-connected layer may be removed entirely and latent variables may be collected from the global pooling layer.

In various embodiments, first order features may refer to any derived features calculated from raw data inputs. In various embodiments, such features include but are not limited to calculations of moving averages, time differencing, detrending, digital signal processing functions (e.g., spectral power analysis, time frequency domain analyses, and/or Fourier transformation), and metrics calculated from logic and/or mathematical computations based on clinical subject matter expertise. These features and measures are described further below with examples provided.

In various embodiments, features may be derived from clinical subject matter expertise. In various embodiments, first order features may be defined by clinicians. In various embodiments, first order features may relate to a subjective or objective rating of a patient's ability to complete a particular task. In various embodiments, first order features may be particular calculations performed on raw health data based on clinical subject matter expertise. For instance, a subject may be asked to listen to three words being spoken then repeat them in order. In various embodiments, a mobile application may record the subject's response as raw audio signal data. In various embodiments, the raw health data may be transcribed to words (e.g., using automatic speech recognition (ASR)) and metrics may be calculated such as: number of words the subject was able to recall; and whether the words in the correct order. In various embodiments, such calculated metrics may be a combination of logic and mathematical operations defined on the raw data as informed by the clinical subject matter expertise of clinicians (e.g., neurologists) who design both the assessment for collecting the data, and the manner in which to measure subject responses to produce a metric.

In various embodiments, additional examples of first order features (using the above speech example) that are based on clinical subject matter expertise include but are not limited to immediate recall; delayed recall; the time taken to recall each word; the accuracy of words recalled; number of hesitations when recalling; errors while recalling the words; words recalled with and without cueing; voice volume, tone and/or pitch; dysarthria (difficulty forming words), speech disorder, and/or vocal tremor.

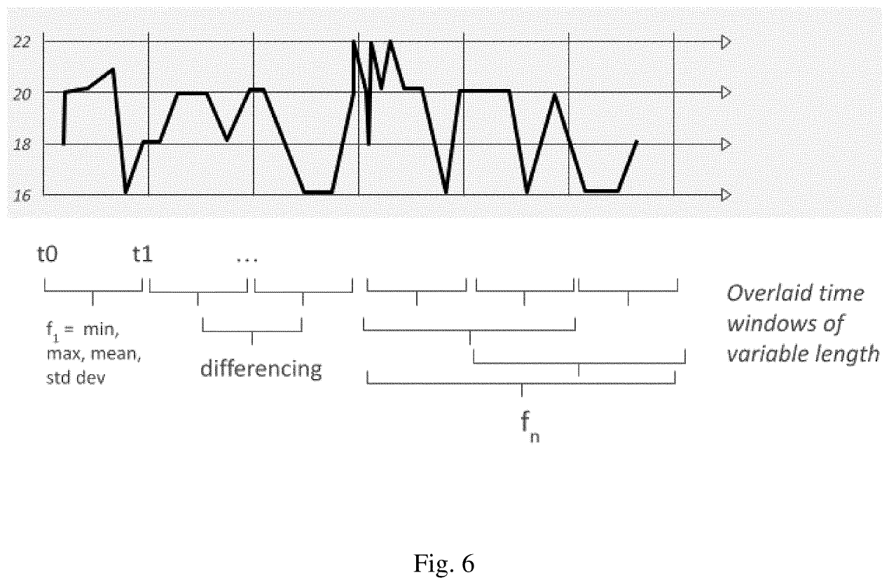

FIG. 6 illustrates a method of calculating a time-windowed aggregation. In various embodiments, features may be derived from data-driven calculations and/or transformations. In various embodiments, time-windowed aggregations such as moving aggregations, local or global minimum, local or global maximum, and/or standard deviation can be calculated on any raw temporal data time window. An example is shown in FIG. 6. In various embodiments, a time window is selected. For example, a time window of one second for the notional data shown in Table 1 may be selected. In this example, a time window of one second would produce 240 values for each dimension within each time window. For each of those samples within a single time window, statistics (e.g., mean, min, max, and standard deviation) may be determined. In various embodiments, determining statistics for each time window may transform the raw data into these aggregate values on a second by second basis. In various embodiments, a longer or shorter time window may be selected. In various embodiments, any suitable amounts of time by which the window is shifted may be used when calculating these time-window aggregations. In various embodiments, moving averages, decay functions, and/or smoothing functions may be applied to the raw health data. In various embodiments, these methods may be applied recursively with any number of overlaid time windows of variable length and the output of smaller time windows may be aggregated within larger time windows. In various embodiments, these values can be z-scored to provide robust information even when used in different scenarios.

In various embodiments, time differencing can be applied either to the raw data or the output of time windowed aggregations, as described above and shown in FIG. 6. In various embodiments, the value at one point in time (e.g., raw data, mean, max, standard deviation, etc.) may be subtracted from the value at another point in time on a given interval and this process may be repeated for each data point. In various embodiments, the output of this operation may be a new time series composed of differences between values of the old time series. In various embodiments, these methods may be used for detrending data to make it stationary for various modeling purposes. In various embodiments, other suitable methods for detrending may include smoothing functions, moving averages, and regression analysis. In various embodiments, these methods produce a time series output which may be a transformation of the raw data and can be provided as input to various machine learning models such as the one shown in FIG. 5.

In various embodiments, second order features may be determined from the raw data and the first order features. In various embodiments, empirical second order features may correlate with first order features. In various embodiments, many measures in clinical care are observational in nature and can generally be referred to as signs and/or symptoms of disease. In various embodiments, some signs and/or symptoms may be evaluated with biomarkers in an objective and quantifiable manner using specific devices and testing procedures. In various embodiments, some symptoms cannot be evaluated in such a direct manner and must be evaluated by a professional with clinical subject matter expertise. One example is the diagnosis of mild cognitive impairment, which requires that the individual have cognitive deficits that are perceived to interfere with their activities of daily living. As such, this determination relies heavily on discussion with the individual, family and caregiver to definitively reach such a diagnosis. Other examples are motor disorders such as essential tremor, or tremor associated with Parkinson's disease, which are shared by the individual during a clinician visit, and observed and noted by the physician. In various embodiments, a rules system and library of templates may be set up to thereby generate second order features even if they cannot be directly evaluated in an objective and quantitative manner In various embodiments, information for populating values for these features may be directly assigned by clinicians from observations, or parsed and processed from clinician notes using natural language processing techniques on electronic health records.

In various embodiments, human-produced features may be combined with labels from clinical subject matter expertise with machine learning. In various embodiments, clinical research may indicate that certain measures are predictive of neurological function. For instance, frailty may be considered a predictor for post-operative delirium. In the example of predicting delirium, in various embodiments, clinicians may want to consider some measure of frailty in addition to other factors. In various embodiments, frailty may itself be a composite of several other measures considered together. In various embodiments, a first machine learning model may be trained to predict a target variable measure, such as frailty, that is defined from logic and/or math constructed from clinical subject matter expertise. A second machine learning model may be trained to receive, as input, the measure from the first machine learning model as a feature input while being trained to predict a higher-level target variable such as risk of delirium.

In various embodiments, a workflow for applying supervised learning may include the following general processing steps: 1. Clinicians label patient records with a score for frailty based on their subject matter expertise. In various embodiments, labels can also be derived from biomarkers known to correlate with frailty that are available in patient health data. In both cases, clinicians provide the logic and calculations for assigning label values. 2. Additional multimodal data may be associated with each subject such as the subject's performance on tasks and/or assessments. In various embodiments, the data associated with a subject would include multimodal inputs as well as the target variable of frailty to predict using those inputs. 3. Supervised machine learning models are trained on the multimodal input data to predict the frailty target variable. 4. When new subjects are evaluated, even if clinicians had not labeled their records with a score for frailty, the newly trained machine learning model can be used to predict a frailty measure for that subject. 5. A real or predicted frailty measure produced from the model can then be used as a second order feature in a subsequent machine learning models (e.g., a pre-trained delirium model provided by a third party) or a rule systems for predicting a new target variable such as risk of delirium, and/or making a recommendation for an intervention.

In various embodiments, certain machine learning algorithms may learn latent variables by being trained on a general task, in an approach known as transfer learning, wherein the latent variables can then be used to encode raw data as second order features to be used as input to a secondary model trained for a different task. One example of transfer learning is the pre-training of a neural network on a general task and then using and updating the resulting model with additional training on a different task. A specific example of this approach is the BERT deep transformer model.

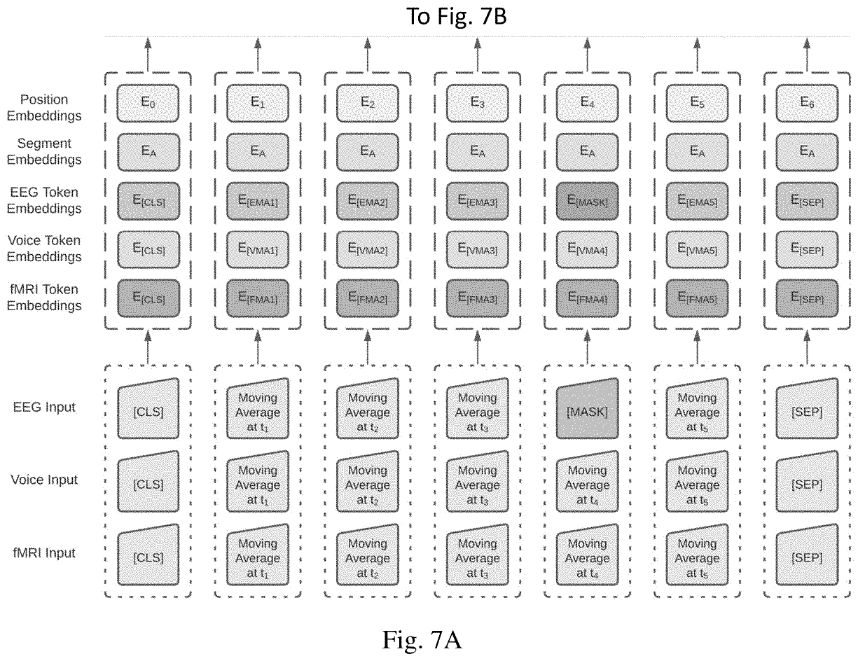

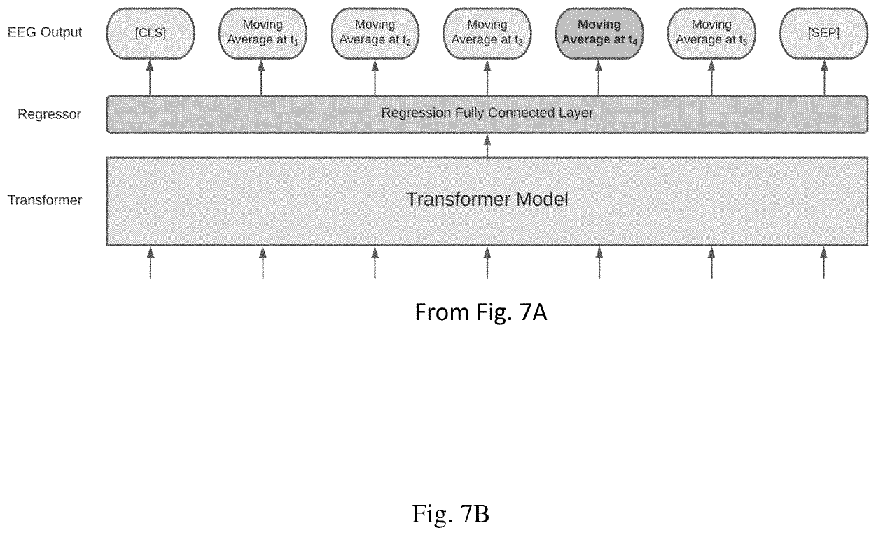

FIGS. 7A-7B illustrate a machine learning workflow for synthesizing missing data points of health data within a time series. In various embodiments, a machine learning model may be provided health data and/or first order features from a plurality of modalities (e.g., moving averages of EEG values, recorded audio/voice, and fMRI images). In various embodiments, any suitable forms of multi-modal health data may be collected while a patient performs tasks or assessments or another general activity (e.g., exercise). In various embodiments, the data inputs may be from any of the above-described data sources or modalities. In this example, EEG data is collected on a subject while they perform an assessment. In various embodiments, this data may be collected on a large population of patients, creating a large library of such data. In various embodiments, a machine learning model (e.g., recurrent neural network) may be trained where the EEG data is the input. In various embodiments, the trained model may be used to synthesize missing values in other patients' EEGs.

In various embodiments, EEG data may not be complete—for example, some values may be missing or corrupted (e.g., due to patient motion or electromagnetic interference). In various embodiments, the machine learning model may learn embedding for each time window of each modality, segment embeddings for each time window, and/or position embeddings for each time window. In various embodiments, incomplete patient EEG values may be supplied to the trained model input. In various embodiments, data from other modalities (e.g., eye tracking, voice, fMRI, etc.) may be supplied to the trained learning system. In various embodiments, the output of the model may predict the value for the missing data in the modality where data is missing. For example, as shown in FIGS. 7A-7B, moving averages of EEG data may be provided where there is a set of values at every second for every 5 seconds. In various embodiments, where an average for one of those seconds is missing, the trained model may predict that value based on the prior training on a plurality of patients' health data and/or first order features. In various embodiments, the model may learn the general sequence of EEG data and establish a sort of “language model” for EEGs.

In various embodiments, the final output layer of the model may be removed and the output of an intermediate hidden layer may be used as second order features used in other models. In various embodiments, the embeddings learned in this process, and the hidden layers may be used as latent variables or second order features that can be used for other tasks. For example, a new output layer for prediction MOCA scores may be added to the trained model. In various embodiments, embeddings may be generated for future EEG data and these embeddings may be fed into other machine learning algorithms (e.g., a third party-supplied model) such as support vector machines for prediction MOCA scores.

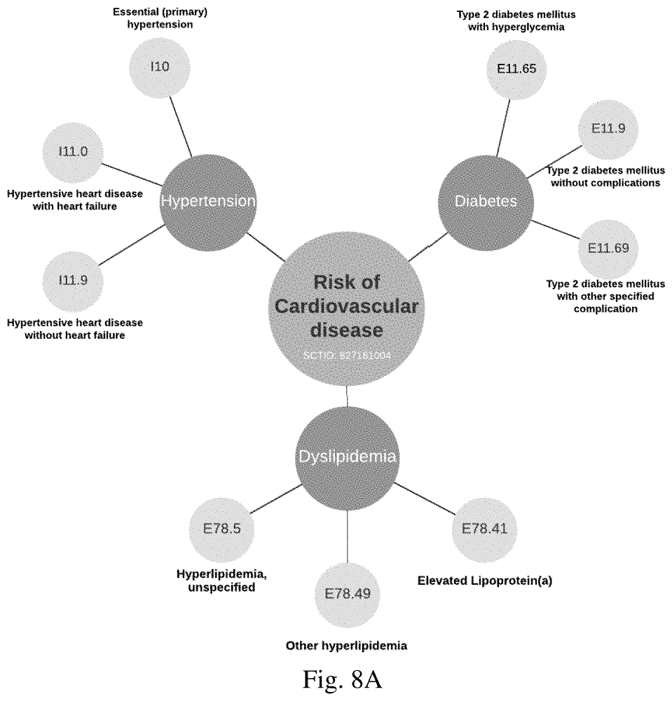

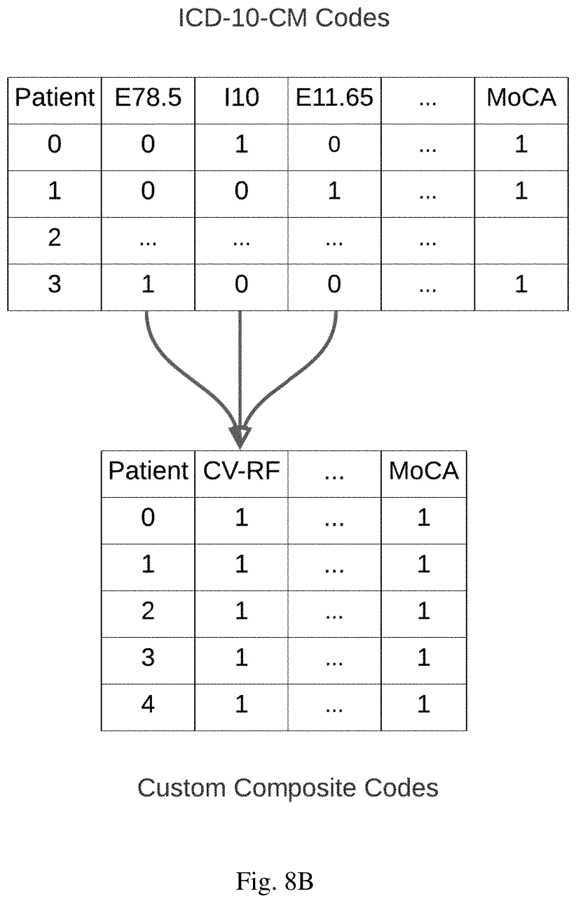

FIGS. 8A-8B illustrate an exemplary clustering of disease codes. In various embodiments, health data may be human-produced from subject matter expertise. In various embodiments, clinicians may use subject matter expertise to group features into aggregate features that carry more predictive power than the individual features alone. FIG. 8B shows an example where several related ICD codes are one-hot encoded, i.e., they are assigned a value of 1 if this code appears in the patient's medical record, and 0 if not. In various embodiments, this method of representing medical codes can serve as input for machine learning algorithms In various embodiments, because the codes in the table at the top of FIG. 8B are all related to complications of cardiovascular health, the codes can be grouped together into one custom code that is then one-hot encoded to 1 if any of the constituent codes appear in the patient's medical record. In various embodiments, the result is shown in the table at the bottom of FIG. 8B, where several ICD codes related to cardiovascular disease from the table at the top of the image have been mapped into one custom code, CV-RF in the bottom table.

In various embodiments, representing features this way may result in more dimensions being used as input to the machine learning algorithm. In various embodiments, increasing dimensions can lead to a problem called the curse of dimensionality wherein the parameter space grows exponentially, effectively diluting the predictive power of any individual feature. In various embodiments, a clinician may be aware that the ICD codes provided here are all related and can be considered as an aggregate. By combining these disease codes, the number of dimensions may be reduced, thus reducing (e.g., minimizing) the curse of dimensionality, while increasing the predictive power of the features provided to supervised learning algorithms.

In various embodiments, the system may include an ontology mapping related to its rules engine that enables clinicians to group first order measures such as ICD codes into second order features such as the defined aggregate code shown in FIG. 8B. In various embodiments, this particular ontology is a grouping of ICD codes related to cardiovascular health into a single aggregate code which is particularly relevant as a comorbidity for assessing risk for various neurological issues when combined with other factors. FIG. 8A shows the specification of this one ontological component which is the aggregate code representing cardiovascular risk factors, including include hypertension, diabetes, dyslipidemia, obesity, smoking, poor nutrition, physical inactivity among others. In various embodiments, cardiovascular risk factors such as hypertension and diabetes may be key risk factors for developing age-related cognitive decline and dementia, and many of these cardiovascular risk factors may be found in the same person. Current estimates indicate that 1 in 3 adults in the US have hypertension and nearly 80% of individuals with diabetes also present with hypertension. In various embodiments, by clustering such individual factors and contextualizing them with other sources of data (e.g., genetic, behavioral, performance-based assessments and other types of health data), machine learning algorithms will enable improvements in the predictive ability to estimate risk for cognitive impairment and dementia, as well as responsiveness from targeted interventions.

In various embodiments, clinical subject matter expertise can be used to group first order features and/or second order features into aggregates that are more predictive for another use case of prediction. Some additional examples include but are not limited to: 1. Grouping ICD codes as PHE codes, to distinguish a particular phenotype. They are mainly used to eliminate case contamination of control groups. PHE codes also define exclusion codes to prevent contamination by cases in the control group. 2. Conceptual groupings of drugs based on chemical composition or physiological effects, such as broad spectrum antibiotics considered as a group. 3. Medi-Span Generic Product Identifier (GPI) to group drugs in classes or subclasses based on therapeutics of the drug. The first 6 characters in a GPI are known as level 6 codes, and are used to identify the therapeutic class of the drug as defined by Medi-Span. 4. Groupings of particular metrics based on current understanding of brain function. For instance, frailty can be defined as a clinical syndrome characterized by age-related decreases in physical, psychological and social functioning. Utilizing the deficit-accumulation clinical model, routinely collected items of a comprehensive geriatric assessment (such as medical history and functional abilities) can be used to compute a frailty index, which gives insights into the degree of frailty for a particular individual.

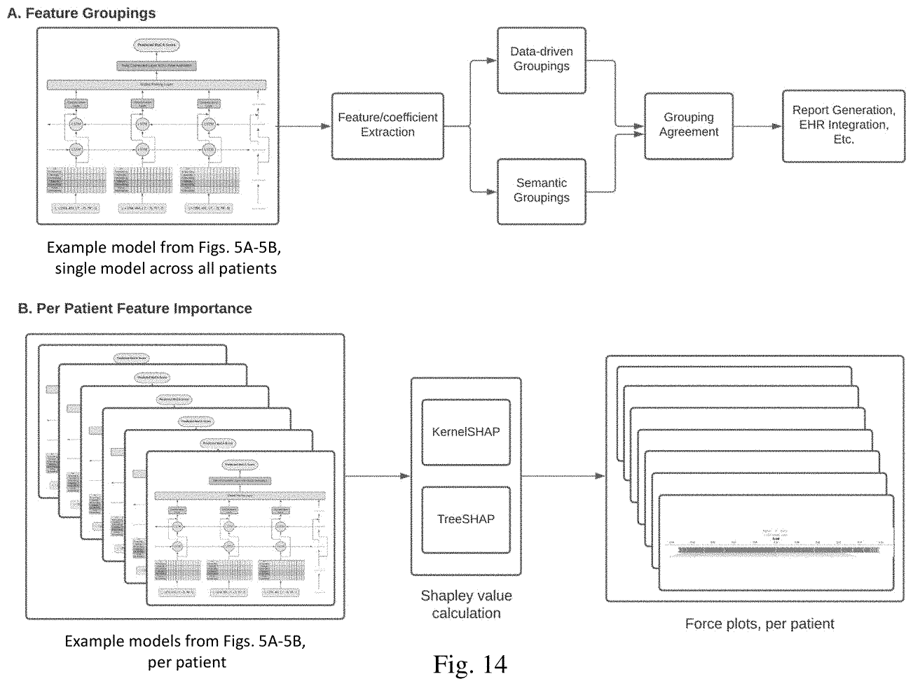

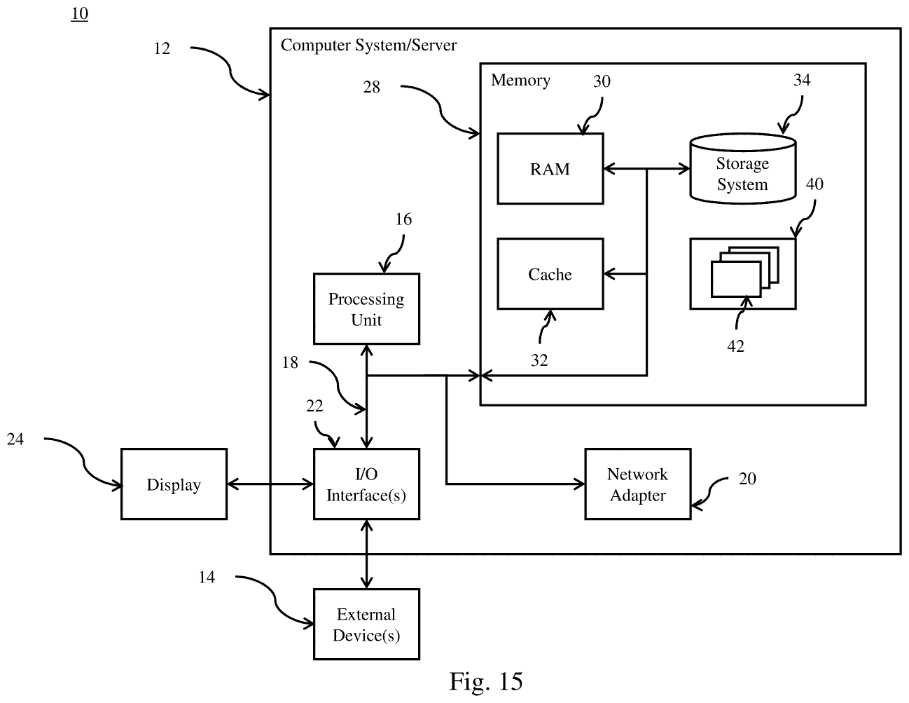

FIG. 14 illustrates an exemplary feature grouping and determination of importance for per-patient features. As shown in FIG. 14, using the exemplary model shown in FIGS. 5A-5B, feature coefficient extraction may be performed. In various embodiments, data driven groupings and semantic groupings may be determined after the feature/coefficient extraction. In various embodiments, agreement may be determined between groupings of the data-driven groupings and the semantic groupings. In various embodiments, the results may be provided for report generation, EHR integration, etc. In various embodiments, data driven groupings may be determined based on clustering, as described throughout the disclosure. In various embodiments, semantic groupings may be determined based on clinical subject matter expertise (e.g., manually or automatically, for example, through rules, combining specific features, metrics, and/or concepts together in an aggregate based on their clinical knowledge).

In various embodiments, the patient models (using the model of FIGS. 5A-5B) for each patient may be analyzed for the importance of the features for each patient. In various embodiments, a Shapley value may be determined for each patient model. In various embodiments, the Kernel SHAP (SHapley Additive exPlanations) algorithm may be applied to the individual patient model(s). The Kernel SHAP algorithm provides model-agnostic (black box), human interpretable explanations suitable for regression and classification models applied to tabular data. This method is a member of the additive feature attribution methods class; feature attribution refers to the fact that the change of an outcome to be explained (e.g., a class probability in a classification problem) with respect to a baseline (e.g., average prediction probability for that class in the training set) can be attributed in different proportions to the model input features. Documentation for Kernel SHAP can be found online at https://docs.seldon.io/projects/alibi/en/stable/methods/KernelSHAP.html. In various embodiments, the Tree SHAP algorithm may be applied to the individual patient model(s). The Tree SHAP algorithm provides human interpretable explanations suitable for regression and classification of models with tree structure applied to tabular data. This method is a member of the additive feature attribution methods class; feature attribution refers to the fact that the change of an outcome to be explained (e.g., a class probability in a classification problem) with respect to a baseline (e.g., average prediction probability for that class in the training set) can be attributed in different proportions to the model input features. In various embodiments, force plots may be generated for each patient based on the determined Shapley value(s).

In various embodiments, clustering of patients may be performed to enable anomaly detection and differential diagnosis. In various embodiments, the general workflow may include: 1. Project all or some subset of the features in the patient data model into vectors; 2. Apply dimensionality reduction techniques such as principal components analysis. 3. Apply a clustering algorithm (e.g., K-means, K-nearest neighbor, DBSCAN, spectral clustering, etc.)

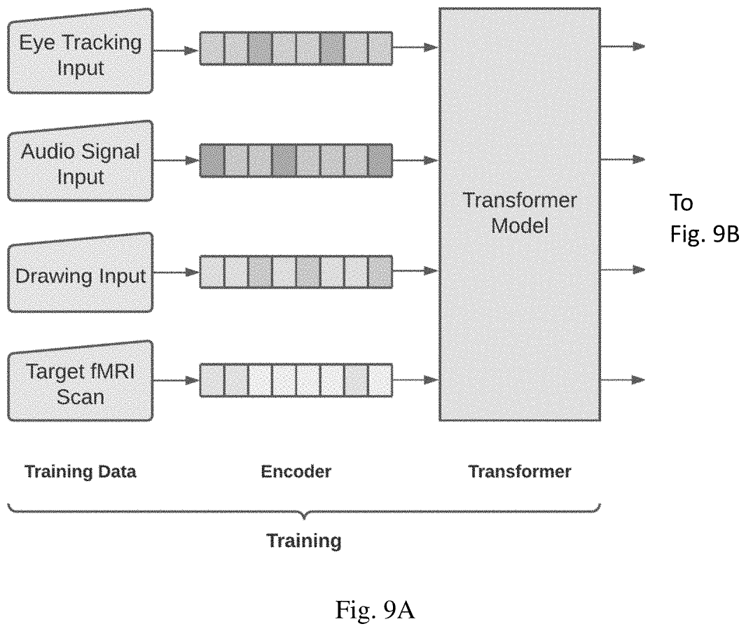

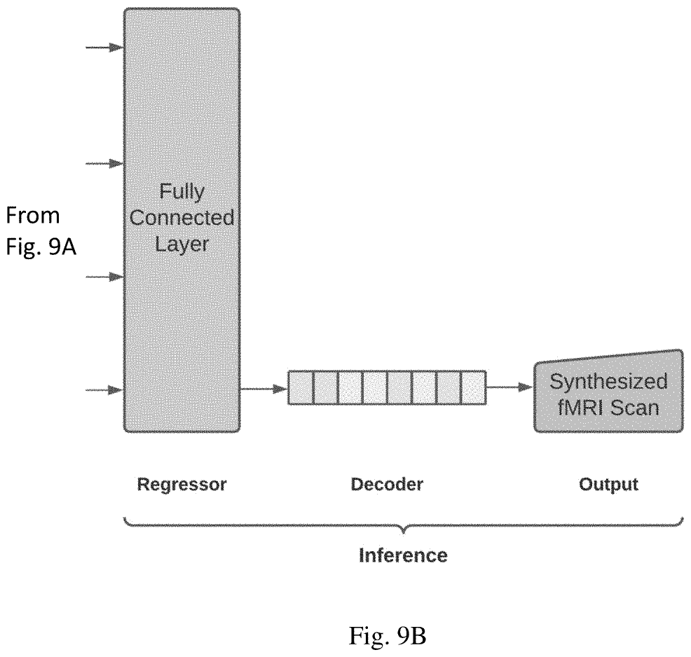

FIGS. 9A-9B illustrate a machine learning workflow for synthesizing missing health data in a modality from a plurality of other modalities. In various embodiments, assessing patient health based on health data can be difficult as the health data may be noisy, missing, or of questionable quality. In various embodiments, collecting multimodal data enables the learning of relationships between the modalities, such as how each modality correlates with other modalities, and how each modality collectively correlates with target variables, such as predicting disease conditions or recommending optimal treatment paths. In various embodiments, the learning of associations between data from different modalities allows for synthesizing of missing data for modalities that were not collected in a patient's health record and/or predicting how interventions may affect one modality using data from another. For example, a drug treatment that affects EEG signals may be used to synthesize fMRI images that are predicted to result from the drug treatment.

In various embodiments, a supervised machine learning approach may be applied to train a model where the data from one modality is used as the target variable and data from one or more other modalities is used as the input features. Different permutations of modalities can be used to predict other modalities. For example, the following multimodal data may be collected concurrently from several patients performing a battery of tasks and/or assessments: 1. Eye tracking data; 2. Voice recording data; 3. Drawing assessment data; 4. EEG data; 5. fMRI taken on for subject during the assessments and then uploaded to the platform.

In various embodiments, raw data from the first four modalities (e.g., eye tracking data, voice recording data, drawing assessment data, and EEG data) as well as first and/or second order features as described above in the sections may be used as training data to synthesize what data collected from an fMRI would look like for a particular patient. In various embodiments, data for each modality is available for the training data. Specifically, in this example, the location of the patient's eye gaze, an audio signal representing what they are saying, their interactions with the mobile device interface while drawing, EEG recordings, and fMRI data of their brain activity. In various embodiments, a machine learning model may be trained with the output of fMRI as a target variable. An exemplary workflow of modeling is shown in FIGS. 9A-9B. In various embodiments, this model may be used to generate synthetic fMRI signals given only eye tracking, voice recording data, drawing activity, and/or EEG data. In various embodiments, generating of synthesized data for one modality when only provided with data from one or more other modalities is particularly useful for future studies or for completing electronic health records, where not all data modalities are available and a user may want to synthesize the missing modalities from those modalities which are present to produce more robust predictions of other target variables such as disease states or to make recommendations for interventions. In various embodiments, a user may generate synthesized health data for a particular missing modality to provide a completed set of inputs to a third party machine learning model trained to, for example, output disease labels based on the missing modality (alone or in combination with the available data modalities).

In various embodiments, fMRI may be correlated with higher order information such as what regions of the brain are being activated in what ways by certain stimuli. In various embodiments, fMRI values may be inferred from the other modalities (e.g., eye tracking, voice, EEG, and/or drawing assessments), and fMRI data can indicate what regions of the brain are being activated. In various embodiments, the regions of the brain being activated can be inferred by the available other modalities and a synthesized fMRI image may be generated based on the other data modalities. In various embodiments, this functionality can be used to test effects of interventions using future assessments by inferring which regions of the brain are being affected and in what ways. For example, clinical trials may indicate that different interventions such as administration of a drug or transcranial electrical stimulation should affect certain regions of the brain in particular ways. During a trial to prove these effects, a clinician could administer the intervention, administer a battery of assessments, collect data from modalities that are easier to collect, and then predict or impute values for fMRI or effect in brain regions (as fMRI is an expensive modality to own and operate with specialized technicians). In various embodiments, the results from the synthesized modality may be useful to indicate whether the predicted values match expected values. In various embodiments, generating synthetic health data from a missing modality from other modalities can be invaluable in situations where it is difficult to collect certain modalities of data but those modalities can be predicted from other modalities.

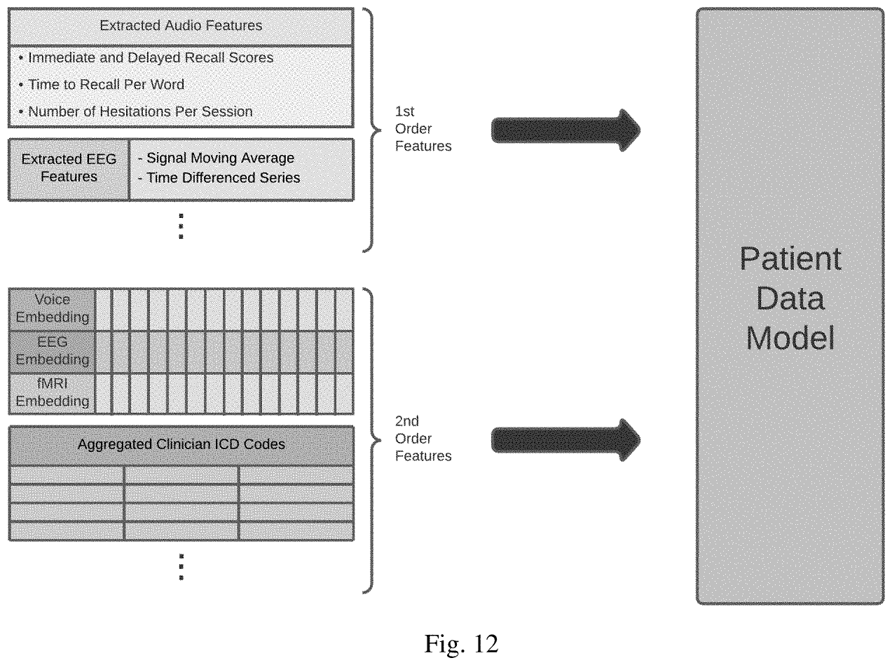

FIG. 12 illustrates an exemplary workflow of a patient data model (a “digital twin”). In various embodiments, a patient data model or “digital twin” may be generated that captures overall health as well as refined relationships between multimodal data representing cognitive functioning. In various embodiments, this model may include the combined features described above including first and second order features, all derived features, features based on clinical subject matter expertise, aggregate features, and every data modality. In various embodiments, a software platform may learn the relationships between these features using statistical approaches and machine learning. In various embodiments, additional profiling may be performed to learn interaction effects between all fields of the patient data model. In various embodiments, missing values may be synthesized in a given patient data model to support various analysis. In various embodiments, some algorithms may be less affected by missing values, and in other cases, the fact that values are missing may itself provide information that is valuable for predicting various patient states. In various embodiments, a model may adapt to these different scenarios depending on the analysis use case at hand.

In various embodiments, the digital twin model may include an index across all of the features along with metadata associated with all of the correlations therebetween. In various embodiments, the metadata may include machine learning models. In various embodiments, the metadata may include statistical models, such as Bayesian models of joint probability distributions across all feature permutations.

In various embodiments, the digital twin model may be particularly useful in that data can be synthesized where it is missing in a patient's medical history. In various embodiments, to be able to synthesize data, source data for creating the synthesizing models may be needed. In various embodiments, in cases where there is no data for a patient, evidence synthesis may be used for randomized control trials to create rules for data synthesizing. In various embodiments, where data is provided for a patient, one or more models may learn the correlations between variables and one or more models (may be different models) may be used to synthesize that data.

In various embodiments, where data is not available for a machine learning model, the latent representations can be determined based on evidence synthesis from RCTs in published literature and our own subject matter expertise from clinical staff. In various embodiments, where data is provided, as described above, machine learning models may be trained on the data. In various embodiments, the model(s) may include neural networks that learn latent representations. In various embodiments, model(s) may include Bayesian generative models trained on joint probability distributions across all of our features (e.g., raw data, first order features, and/or second order features).

In various embodiments, statistics on the interaction effects between all variables may be updated. In various embodiments, machine learning models may be updated. In various embodiments, as new data comes in, one or more distributions may be measured of the data, including how far the new data drifts from the data that the current statistics and machine learning models are based on. In various embodiments, a drift threshold may be determined for determining when to trigger and update of a particular model.

In various embodiments, the patient data model captures not only all raw data and features described herein also but the relationship between each feature. In various embodiments, this “digital twin” may be used to represent subject brain physiological state as well as a composite of metrics that represent the overhaul state of cognitive health for a subject as defined by the various metrics and biomarkers described herein.

In various embodiments, the construction of the “digital twin” of a patient can serve several purposes including but not limited to: 1. Using the patient data model states and variables as input to predicting disease conditions; 2. Using patient data model states as the inputs to an optimization algorithm for recommending interventions; 3. Using the patient data model states and some assessment of their value as an objective function in reinforcement learning for recommending interventions; 4. Using the patient data model to predict or detect effects of an intervention such as drug administration when only limited data modalities for measurement are available.

In various embodiments, algorithms for predicting and detecting disease conditions may differ from those for optimizing recommendations for interventions, and potentially those for differential diagnosis. In various embodiments, the raw data and first and second features described in prior sections can be used for all of these use cases.

In various embodiments, any suitable permutation of the features described above may be used as input to supervised machine learning methods to predict or detect biomarker values or disease conditions. In various embodiments, multiple models can be trained where each model focuses on a different target variable. In various embodiments, potential target variables include but are not limited to Alzheimer's, Parkinson's, amyotrophic lateral sclerosis (ALS or Lou Gehrig's disease), Amyloid protein, Tau protein, and/or State of the digital twin of the patient data model. In various embodiments, the model of FIGS. 5A-5B may be adapted to predict one of these target variables, for example, predicting Alzheimer's via multimodal input that combine first order and second order features, and, optionally, human created inputs. In various embodiments, variables can be time lagged such that the prediction is anticipating the patient state at some time in the future, e.g., 1 year from the current date. In various embodiments, features can also be targeted for prediction or detection within a shorter period of time more for immediate diagnostic purposes.

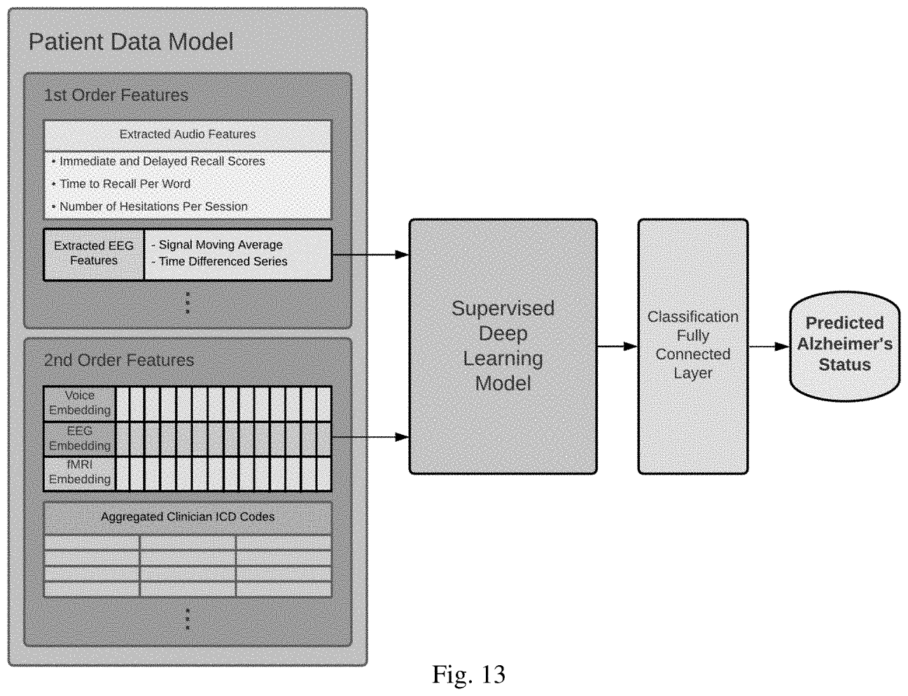

FIG. 13 illustrates an exemplary model leveraging first and second order features to predict the onset of Alzheimer's disease. In various embodiments, variables can be time lagged such that the prediction is anticipating the patient state at some time in the future, e.g., 1 year from the current date. In various embodiments, features can be targeted for prediction or detection within a shorter period of time more for immediate diagnostic purposes. In various embodiments, the patient data model may include first order features (shown in FIGS. 12 and 13). For example, the first order features may include extracted audio features, such as immediate and delayed recall scores, time to recall per word, number of hesitations per session, etc. In another example, the first order features may include extracted EEG features, such as signal moving average and time differences series. In various embodiments, the patient data model may include second order features (shown in FIGS. 12 and 13). For example, the second order features may include learned embeddings from a neural network (e.g., RNN), such as voice embedding EEG embedding, fMRI embedding, etc. In another example, the second order features may include aggregated clinician ICD codes.

In various embodiments, rules may be defined and applied to combine output of predictive models with other criteria that may drive clinical decision support. In various embodiments, using machine learning to predict the likelihood a subject may have Alzheimer's disease may be a driver of clinical decision-making, but may need to be considered along with other criteria when providing information to a clinician for making decisions. In various embodiments, additional criteria may include but is not limited to: particular populations treated at specific locations that may modify interpretation of machine learning model output which was trained for the general population; subjects being under 18 years of age wherein outlier variables may skew machine learning results but a diagnosis of certain neurological conditions would not be appropriate; user preferences wherein clinicians at a particular locations prefer higher recall at the cost of precision or vice versa when predicting disease conditions; integration into clinical workflows, where predictions must be translating into particular categories for clinical follow up within the context of the treatment center and normal operations.

In various embodiments, a rules engine may be provided where clinicians author rules to combine situational criteria as described above with output of machine learning models as well as first and/or second order features as described above to provide actionable clinical decision support. Moreover, the platform can enable users to author their own custom rules and share rules with others to achieve consensus on best practice.

In various embodiments, features used as input may be grouped. In various embodiments, the groupings themselves are not necessarily used as features for input to the machine learning algorithm. In various embodiments, the groupings may be semantic groupings of the features that convey a more semantically meaningful interpretation of how their values influenced the model prediction output.

In various embodiments, the machine learning models described herein may be used for anomaly detection in health data of a patient (e.g., an EHR). In various embodiments, it is not always possible to predict a specific disease condition for a subject, but it may still be meaningful to evaluate the degree to which a subject deviates from what is considered normal. In various embodiments, one or more machine learning models may be included to analyze patient health data and determine where values deviate from the norm. In various embodiments, normal values may be defined from clinical guidelines or standards. In various embodiments, an exemplary workflow is as follows: 1. Create a data set consisting of instances of a patient data model (e.g., a digital twin model) with only subjects that have not been diagnosed with neurological issues (i.e. who are “health”); 2. Run clustering as described in the prior section on this data set. In various embodiments, this will naturally classify patients into groups in a data driven way. In various embodiments, groups may be based on age, gender, or any of the features described above; 3. Train a one-class classification model using a method such as support vector machines. In various embodiments, the same vectors projected from the patient data model can be used as input to this model training. 4. Ingest data related to the new subject to be evaluated and create a patient data model (it is entirely acceptable to have missing data); 5. Find the nearest cluster to the new subject and look up the one-class classification models associated with it. 6. Evaluate the patient data model vector associated with the new subject using the one-class classification model to determine if the new subject is considered part of that class or not. If not, then the new subject is an outlier and deviates from normal neurological functioning.

In various embodiments, the platform may support differential diagnosis and can recommend assessments based on its results. In various embodiments, clinicians may enter values for first and/or second order features associated with symptoms as described above. In various embodiments, a library of rules may be provided for evaluating features to recommend particular assessments based on their values. For example, if a subject has several symptoms that are common to Alzheimer's disease, the system may process a rule that suggests the application of one or more assessments specifically designed for measuring likelihood of Alzheimer's such as a clock drawing assessment. In various embodiments, machine learning can be used to drive assessment suggestions based on the results of comparing new subjects with existing clusters. In various embodiments, a workflow may include: 1. Create a data set consisting of instances of the Linus patient data model with subjects that have been diagnosed with different neurological issues (e.g. Alzheimer's, ALS, Parkinson's, etc.); 2. Run clustering as described in the prior section on this data set. This will naturally classify patients into groups in a data driven way. It is unlikely the resulting clusters will all include subjects with one of the conditions mentioned in the first step. Instead, each cluster will likely have some subset, with one condition serving as the majority case; 3. Ingest data related to the new subject to be evaluated and create a patient data model instance for them. It is entirely acceptable to have missing data; 4. Find the nearest cluster to the new subject; 5. Enumerate the conditions represented by any subjects within the nearest cluster; 6. Suggest a collection of assessments determine either by the majority condition of the cluster or some subset of conditions.

In various embodiments, recommendations may be made using optimization algorithms which differ from those used for the purposes of diagnosis and prediction, though they may operate on the same features. In various embodiments, recommendation engine may analyze the current condition, the possible actions to take, and optimize for the action which will most likely produce the desired effect for changing the current condition. In various embodiments, the recommendations engine may apply the health data synthetization models as described above to analyze potential results of a particular treatment for a patient (e.g., using the patient digital twin data model).

In various embodiments, within the context of the defined system, the current condition of the patient may be predicted using the methods described above, but can also come from direct measurements where possible.

Evidence-based medicine is a process that integrates expert clinical knowledge, the highest available scientific evidence, and patient values, desires and needs to guide decision-making involved in clinical management. Best practices in clinical knowledge may be sourced from different origins, best diagnostic practices may be sourced from clinical practice guidelines disseminated by professional organizations (such as the American Academy of Neurology or American Heart Association), and best interventional practices are sourced from the highest available evidence, which is graded on a scale that ranges from I to VII (lower indicating strongest evidence level), as seen in Table 4 below.

TABLE 4

| Evidence | |
|---|---|
| Level | Source |
| I | Evidence from a systematic review of all relevant randomized |
| | controlled trials (RCT's), or evidence-based clinical practice |
| | guidelines based on systematic reviews of RCT's |
| II | Evidence obtained from at least one well-designed RCT |
| III | Evidence obtained from well-designed controlled trials |
| | without randomization, quasi-experimental |
| IV | Evidence from well-designed case-control and cohort studies |
| V | Evidence from systematic reviews of descriptive and |
| | qualitative studies |
| VI | Evidence from a single descriptive or qualitative study |
| VII | Evidence from the opinion of authorities and/or reports of |
| | expert committees |

In various embodiments, clinicians may define rules that may take in several inputs including but not limited to: Highest available evidence; Patient desires, needs and individual preferences; Prediction of biomarker values from methods described in prior sections along with feature importance in determining the various predicted values; Raw data from electronic health records and multimodal assessments; first order measures calculated from multimodal assessment data; second order measures (latent variable representations); and/or Clinical settings. In various embodiments, the rules may apply logic which combines these input values into an output recommendation based on clinically established best practices.

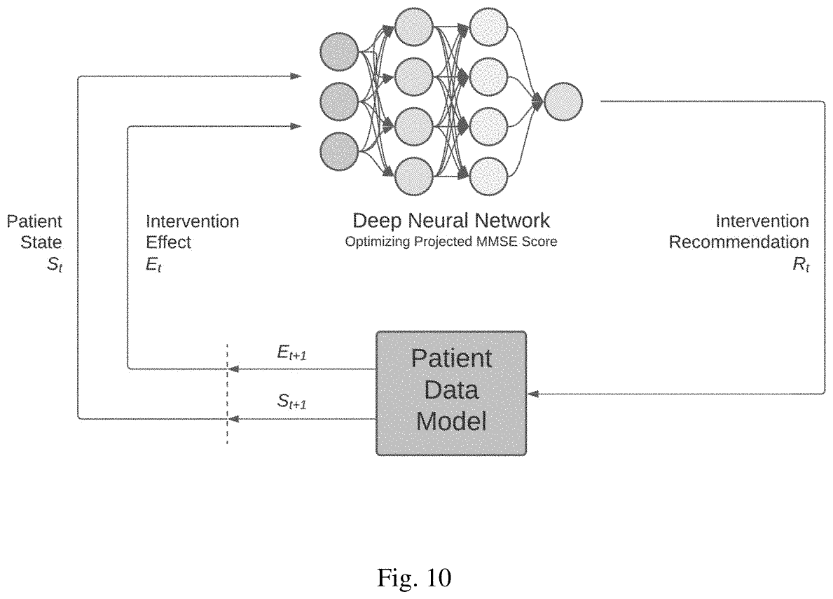

In various embodiments, reinforcement learning can be used to train a model that seeks to provide optimal intervention recommendations. In various embodiments, Deep Q Learning may be used, as shown in FIG. 10, which works by leveraging a pair of neural networks to forecast the effects of various intervention actions that can be taken, then recommending the action predicted to offer the most benefit. An example of using Deep Q learning within the described platform may receive, as input, raw health data, first order features, and/or second order features. In various embodiments, these features may represent the current state of the subject (e.g., the digital twin). In various embodiments, possible interventions may include but are not limited to: Transcranial electric stimulation; Drug administration or specific titration of dosage; Lifestyle change recommendations such as changes to diet or exercise.

In various embodiments, historical data of these interventions can be used to augment the model's understanding of their potential effects and benefits in certain circumstances. For example, data on subjects receiving transcranial electric stimulation including their performance before and after treatment may be available as well as measures of biomarkers and electronic health data. In various embodiments, the predictive model component of the Deep Q learning model would learn to predict new values for the input features given the intervention based on past data measurements before and after such an intervention. In various embodiments, this process may be repeated for each potential intervention. In various embodiments, the optimization component of the Deep Q learning model would then maximize the objective function to take the intervention that produces the best new predicted values of the input features. In various embodiments, the patient models may be used for predicting a biomarker such as MOCA as the objective function for an optimization algorithm. In various embodiments, in the deep-q-learning case, the models implicit to the optimization process predict the patient state using our patient data model referenced above. In various embodiments, the patient data model may be used as input to a predictive model to predict the MOCA score. In various embodiments, the prediction model may act as the objective function, or a component therein. For example, the optimization algorithm may maximize the MOCA score.

Within any health system, populations and the data collected for the population may change over time. In various embodiments, the machine learning models as well as the rules may be updated when the statistical or logical implications of changes in healthcare and populations deem it necessary. In various embodiments, automated components may be provided to update models and/or rules. In various embodiments, auditing functions may be provided to audit performance of models and/or rules over time.

In various embodiments, machine learning models may be versioned, tracked, and regularly audited using cross-validation to track various measures over time. In various embodiments, these measures may include receiver characteristics, area under the curve (AUC), recall, precision, and/or F1 scores. In various embodiments, once models have reached a certain threshold of model drift, automation will send out notifications and trigger automated updating of models. In various embodiments, automated updating may use more recent data (e.g., data which has caused the model drift) along with samples from older data. In various embodiments, the models may be re-trained with new training data including the recent data.

In various embodiments, different modeling techniques can be used and the AUC of each model compared to identify whether different algorithms would be advised. In various embodiments, supervised learning models capable of handling the number and type of dimensions generated may be used. In various embodiments, model training and evaluation may have the data and hyperparameters versioned such that the models can ultimately be compared against all other updated model versions prior to marking which achieves optimal performance and accuracy as define by the metrics indicated. In various embodiments, models may not be immediately rushed to production but run through a thorough development operations testing processes to ensure that new models not only meet criteria of machine learning metrics, but also do not adversely impact the operational system. In various embodiments, manual checks and audits can also be instituted prior to deployment of updated models. In various embodiments, due to regulation by government bodies, any modeling changes may need to be filed and reviewed prior to deployment.

In various embodiments, rules may be manually updated over time through human intervention given new data trends and as clinical subject matter expertise and best practices evolve. In various embodiments, rules may go through automated quality assurance testing. In various embodiments, these processes may run synthesized and/or real data through rules to comprehensively determine all potential output for every potential input. In various embodiments, further analysis will be performed to identify most likely outcomes based on most likely inputs.

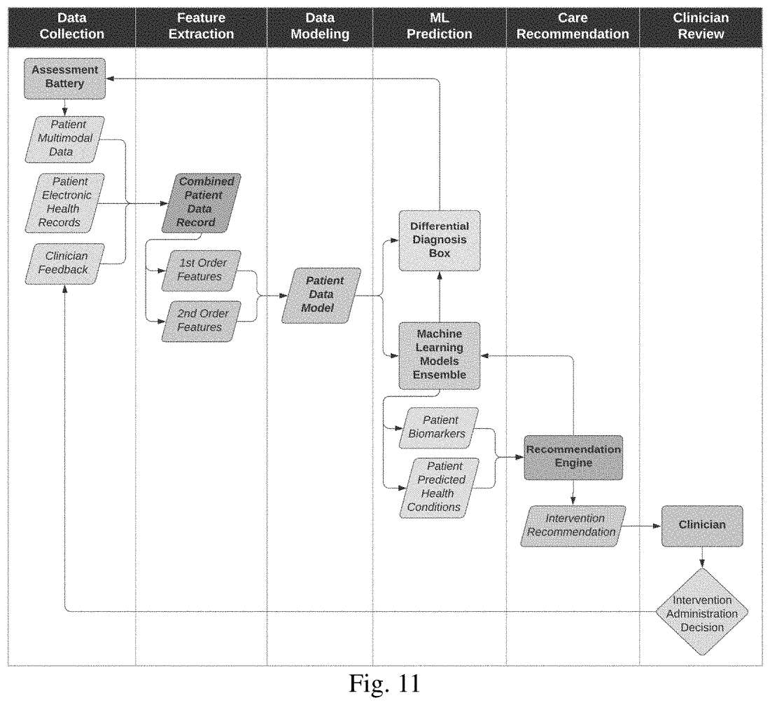

FIG. 11 illustrates a workflow showing a feedback loop of determining clinical recommendations based on patient health data for clinician review. In various embodiments, the methods described above can be combined in an iterative loop for assessing, predicting, and optimizing patient outcomes. In various embodiments, outputs of one component may serve as inputs to another component. In various embodiments, a general workflow may proceed as follows: 1. A battery of assessments are administered to a patient and multimodal data collected on their responses; 2. Additional data from electronic health records, clinician feedback, and others is ingested and combined with multimodal assessment data; 3. first and/or second order features are extracted and/or generated; 4. The features are input into pre-trained machine learning models which predict biomarkers and/or health conditions for the subject; 5. The predicted biomarkers, health conditions, and potentially the features are fed into a recommendation engine which considers the state of these features and recommends one or more interventions that it predicts will produce the most desired changes in those predictions and feature values; 6. The clinician may perform the intervention recommended; 7. The assessments may be re-administered to determine if the intervention had the desired effect.

As shown in FIG. 11, raw data and first order and/or second order features feed into a predictive model to produce a particular output. In various embodiments, the model may be interrogated to understand the feature importance and contribution to this particular output. In various embodiments, clinicians may group the features into semantically meaningful clusters. In various embodiments, when displaying the model output, instead of displaying a single score, the clinician-generated clusters may be provided to the user. In various embodiments, values for the clusters may be calculated as the aggregation of the contributions of their constituent features to the prediction output (e.g., weighted by feature importance).

In various embodiments, the system can leverage various tasks and/or assessments of patients delivered on mobile devices to predict values for other modalities that may be too difficult to directly measure in patients. In various embodiments, the system may use the digital twin data model, in addition to other data to generate synthesized data for other missing or difficult-to-acquire modalities. In one example, a hospital may only have a 2-lead electrocardiogram (ECG/EKG), but a particular machine learning model may require 6 or 12 lead values as input. Machine learning models as described herein may be used to generate synthesized 6 and/or 12 lead data based on the raw 2 lead data, as well as other recorded health data for that particular patient, and first order and/or second order features determined from that data, to generate the synthesized 6 and/or 12 lead data.

In various embodiments, the disclosed systems can reveal how interventions such as drug administration, different dosages, transcranial electric stimulation, or lifestyle changes affect brain physiology and functioning wherein those effects are only directly measurable by modalities such as EEG. Thus, mobile device assessments may act as new measures of intervention effects.

Exemplary Assessments

The following list includes exemplary tasks, including a brief description of those tasks, which may be used in systems and methods according to the present disclosure. The disclosure, however, is not limited to only the following tasks. Other tasks that measure any suitable physiologic conditions or traits may be used. Each of those other tasks and the below tasks may be used alone or in any combination with one another.

In various embodiments, the tasks and/or assessments may include time and space orientation questions, as vocal responses to spatial and temporal orientation questions from MMSE may provide a basic measure of mental status. Temporal orientation, in particular, has been significantly associated with MMSE decline over time and may reveal greater disparity in AD than IVD or PD.

In various embodiments, the tasks and/or assessments may include sentence complete tasks to assess naming and lexical access. In various embodiments, participants may provide vocal responses to open ended prompts about hope and fear. In various embodiments, qualitative analysis of affect and intonation may provide a window into personality and mental state.

In various embodiments, the tasks and/or assessments may include one or more depression and/or anxiety screens. In various embodiments, a combination of questions from PHQ-4 and GAD-2 may be used to assess mood. Late-life depression may be a risk factor for dementia and affects quality of life (QoL). Thus, in patients with dementia, significant anxiety can reduce QoL, impair activities of daily living, and increase caregiver burden.

In various embodiments, the tasks and/or assessments may include a backward digit span task to assess executive abilities including attention and manipulation of working memory. In various embodiments, a Backward Digit Span Test (BDST) may be used. In various embodiments, a test taker may hears a sequence of four (4) digits and be prompted to repeat them in reverse order. In various embodiments, this task may be repeated two or more times and may include any suitable number of attempts per prompt (e.g., a total of three attempts before the prompt is considered a fail).

In various embodiments, the tasks and/or assessments may include one or more ball balancing tasks to assess motor control and coordination. In various embodiments, a test taker may hold a device parallel to the ground and tilts the screen as needed to keep a virtual ball within a target area. In various embodiments, inertial measurement unit (IMU) sensors may be used to measure reaction time, fine motor control, movement characteristics, tremor, and/or dyskinesia.

In various embodiments, the tasks and/or assessments may include dual tasking to assess frontal resource allocation and cognitive-motor interference. In various embodiments, a test taker may be asked to perform the ball balancing and backward digit span tasks simultaneously. In various embodiments, aggregating the cognitive load from multiple complex tasks provides insight into an individual's cognitive reserve and global executive function.

In various embodiments, the tasks and/or assessments may include delayed subjective recall to assess episodic memory. In various embodiments, a test taker may be asked to recall the responses they had previously provided towards the beginning of the test. In various embodiments, a Philadelphia verbal learning test (PVLT) may be used. In various embodiments, automatic speech recognition (ASR) software may be used to determine the accuracy of the response(s). In various embodiments, the voice of the test taker is analyzed to derive speech metrics such as pause rate, pitch, and/or speed.

In various embodiments, one test or assessment may be interchangeable with another test or assessment. For example, a PVLT may be performed instead of an assessment using the bipolar depression rating scale (BDRS). Conditions for changing one or more tasks and/or assessments may be determined by a health care provider. In various embodiments, automated rules may be provided to look for data from an alternative test or assessment when another test or assessment is not available.

Drawing tasks: A series of drawing-based tablet tests may be administered with a tablet and stylus, or other suitable electronic device. An analysis of the time-stamped drawing signal can be conducted to identify early indications of cognitive change. A tablet application captures, encrypts, and transmits the encrypted data to system servers. These drawing-based tasks can include:

Pre-test: An exercise involving copying waves that is administered before completing the other tablet tests (including DCTclock-tablet) with the goal of making the subject comfortable with drawing using the tablet and stylus.

DCTclock™: a neuropsychological test based on the traditional Clock Drawing Test that may provide a more sensitive measure of cognitive state. The DCTclock test capitalizes on the design of the traditional Clock Drawing Test but uses advanced analytics and technology to evaluate both the final drawing and the process that created it, producing a more robust assessment. The DCTclock test, is cleared to market and uses a digitizing ballpoint pen that, while drawing, also digitally records its position on the paper 75 times a second with a spatial resolution of two one-thousandths of an inch. DCTclock software detects and measures changes in pen position that cannot be seen by the naked eye, and because the data is time-stamped, the system captures the entire sequence of behaviors (e.g., every stroke, pause, or hesitation), rather than just the final result. This enables the capture and analysis of very subtle behaviors that have been found to correlate with changes in cognitive function. These measurements are all operationally defined in code (hence free of user bias) and carried out in real time.

Pathfinding test: A series of mazes to be completed as quickly and accurately as possible.

Symbol test: Keys of symbol-digit pairs are provided followed by prompts with empty boxes where the subject is asked to input the appropriate response as quickly as possible.

Connect test: The subject is instructed to connect a set of circles as quickly as possible according to a pre-established pattern.

Tracing test: The subject is asked to trace a line with both their dominant and non-dominant hand.

Decision making and reaction time tests: Participants can be asked to complete three short cognitive tasks presented on a tablet, or other suitable electronic device. These tasks may be sourced from DANA Brain Vital (Anthrotronix, Inc.), which is an FDA-cleared, modular application that measures cognitive efficiency by tracking subtle changes in cognitive capabilities. DANA assessments are highly sensitive and designed for high-frequency use and focus on accuracy and reaction time—two key elements of cognitive efficiency. Each task takes 1-2 minutes to complete. Subjects can also be asked to complete the PHQ-9 depression screening tool. An iPad application captures, encrypts, and transmits the encrypted data to a HIPAA compliant server.

The tasks included may include:

| Task | Description |
|---|---|
| Simple | A bullseye stimulus appears on the screen, and the test taker |
| Reaction | taps it as quickly as possible |
| Time | |
| Procedural | A number (1, 2, 3 or 4) appears on the screen, and the test |
| Reaction | taker must indicate which number was displayed by tapping |
| Time | either the “1 or 2” button or the “3 or 4” button |
| Go/No-go | A building with six windows is displayed, and either a |
| | “friend” (green) or “foe” (gray) alien will appear in a window. |
| | The test taker must tap the “BLAST” button only when foe |
| | stimuli appear |
| PHQ-9 | Self-reported depression screening tool |

Speech elicitation tasks: A system can use elicitation and analytics systems designed to extract outcome measures as indicators of neurological system function from individuals. Tasks are administered, and voice recordings captured and encrypted through a tablet, smartphone, or other voice-capturing device. Voice recordings are then uploaded to a secure, HIPAA compliant cloud server. Transcripts of the voice recordings are created, and an AI engine analyzes for finite but clinically relevant information. Algorithms apply signal processing and cognitive linguistic analysis to assess speech and fine motor skills and detect subtle changes in cognitive function. Extraction of linguistic and phonetic measures have been shown to correlate to Alzheimer's disease and cognitive function.

Speech and voice assessments may include:

| Task | Description |
|---|---|
| Complex | Participants describe a picture of a complex scene |
| Picture | in their own words |
| Description | |
| Category | Participants name as many items as they can that |
| Naming | belong to a category |
| Object Recall | Participants name a set of objects after a short delay |
| Sentence | Participants read a set of experimentally-controlled |
| Reading | sentences |
| Sustained | Participants hold out a sustained vowel sound (/a/) for |
| Phonation | as long as possible |
| Diadochokinetic | Participants repeat a set of alternating sounds |
| Rate | (/bVtVkV/) as quickly as possible |
| Story Recall | Participants recall a short story both immediately and |
| | after a delay |

Eye tracking-based memory assessments: VisMET (Visuospatial Memory Eye-Tracking Task) is a tablet-based application that passively assesses visuospatial memory by tracking eye movements rather than memory judgements. VisMET offers a sensitive and efficient memory paradigm capable of detecting objective memory impairment and predicting cognitive and disease status. This task is conducted on an iPad, or other suitable electronic device, and monitors a participant's gaze location and gaze patterns as they view repeated images that have been subtly changed between the first and second viewing of the image (for example, an item in the first image may have been deleted in the repeated image). This testing captures full face video recordings which will be kept and de-identified locally at the trial site. De-identified, coordinate level data will then be uploaded and a computerized algorithm will generate gaze position to approximate eye position. Cumulative gaze times, dwell times, and other eye movement parameters serve as the some of the first-order measures.

Gait and balance assessment: Cognitive decline and neurodegenerative diseases have been implicated in gait dysfunction via disturbance of top-down mechanisms and frontal-systems' resource allocation and linked to executive dysfunction. Gait velocity decreases, variability increases, and the ability to multitask while walking (dual-tasking) is impaired as cognition declines and can be risk indicators of dementia progression. These features can be captured using motion sensors, such as accelerometers and gyroscopes on smart devices, and such approaches have been validated against in-lab measures. Dual tasking (e.g., walking or standing while performing a cognitive task) disrupts performance in one or both tasks, and resulting dual-task costs have been shown to increase with aging and be reliable indicators of loss of cognitive reserve and development of cognitive dysfunction and early dementia. Specifically, dual tasking activates a network of brain regions, including prefrontal cortex, and is associated with degeneration of the entorhinal cortex. It offers a sensitive quantitative metric of integrity of frontal systems that correlate with executive function and serve as early biomarkers of meso-temporal memory systems. This task is conducted using a study provided smartphone carried in a pocket or phone carrier attached to the subject's waist, or other suitable electronic device. In the gait assessment, the subject is asked to walk at a comfortable pace of their choosing for 45 seconds. They are then asked to repeat that walking exercise while performing a serial subtraction task. The total time walking is <2 minutes. In the balance assessment, the subject is asked to stand as still as possible for 30 seconds with their eyes open. They are then asked to stand for 30 seconds with their eyes closed, and finally, to stand for 30 seconds with their eyes open while performing a serial subtraction task. Total standing time is <2 minutes. Data from these tasks includes gyroscope and accelerometer readings.

Lifestyle questionnaires: In various embodiments, a patient may be asked a series of questions relating to their lifestyle. In one example, the patient may be administered an activities of daily living (ADL) questionnaire. In another example, one questionnaire is adapted from the Barcelona Brain Health Initiative and includes up to 57 yes/no questions about the participant's lifestyle that are associated with cognitive performance. These questions are presented on a tablet, or other suitable electronic device, and the subject uses their finger to select yes or no for each question.

Referring now to FIG. 13, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 13, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of determining one or more biomarker and/or health condition of a target patient, the method comprising:
receiving, as input to a pre-trained artificial neural network, a plurality of health data of the target patient and/or a plurality of first order features determined from the plurality of health data of the target patient, the plurality of health data of the target patient derived from a plurality of modalities;
receiving, from an intermediate layer of the pre-trained neural network, a plurality of latent variables based on the plurality of health data and plurality of first order features of the target patient, and
providing the plurality of latent variables to a pre-trained learning system, the pre-trained learning system trained to receive as input the plurality of latent variables and output one or more biomarker and/or health condition of the target patient.

2. A method of generating a digital model of a target patient, the method comprising:
receiving, as input to an artificial neural network, a plurality of health data of the target patient and/or a plurality of first order features determined from the plurality of health data of the target patient, the plurality of health data of the target patient derived from a plurality of modalities; and
training the artificial neural network to generate, at an intermediate layer thereof, a plurality of latent variables based on the plurality of health data and/or plurality of first order features of the target patient.

3. A method of training a system to determine one or more biomarker and/or health condition of a target patient, the method comprising:
receiving, as input to a first artificial neural network, a plurality of health data and/or a plurality of first order features determined from the plurality of health data, the plurality of health data derived from a plurality of modalities;
training the first artificial neural network to generate, at an intermediate layer thereof, a plurality of latent variables based on the plurality of health data and/or plurality of first order features;
training a second artificial neural network to output one or more biomarker and/or health condition based on the plurality of latent variables.

4. A method of synthesizing health data of a target patient, the method comprising:
receiving, as input to a pre-trained artificial neural network, a plurality of health data of the target patient and/or a plurality of first order features determined from the plurality of health data of the target patient, the plurality of health data of the target patient derived from a plurality of modalities;
receiving, from an intermediate layer of the pre-trained artificial neural network, a plurality of latent variables based on the plurality of health data and/or plurality of first order features of the target patient,
providing the plurality of latent variables to a pre-trained learning system;
providing the plurality of health data and/or the plurality of first order features to the pre-trained learning system, wherein the pre-trained learning system trained to receive as input the plurality of latent variables and at least one of the plurality of health data and/or the first order features, the pre-trained learning system configured to synthesize at least one value associated with the plurality of health data and/or the first order features.

5. The method of claim 1, wherein the one or more biomarker and/or health condition comprises a Montreal Cognitive Assessment (MoCA) score.

6. The method of claim 1, wherein the one or more biomarker and/or health condition comprises a disease label.

7. The method of claim 1, wherein the plurality of health data comprises temporal data.

8. The method of claim 7, wherein the temporal data comprises at least one of: time-stamped coordinates of a limb of the target patient, eye-tracking coordinates of the target patient in response to a visual stimulus, audio signals from the target patient in response to an audiovisual stimulus, pulse data of the target patient, oxygen saturation data of the target patient, blood pressure data of the target patient, and/or electroencephalography (EEG) data of the target patient.

9. The method of claim 1, wherein the plurality of health data comprises non-temporal data.

10. The method of claim 9, wherein the non-temporal data comprises at least one of: blood type of the target patient, genetic phenotyping of the target patient, handedness of the target patient, and/or allergies of the target patient.

11. The method of any one of claim 1, wherein the plurality of first order features are determined by aggregating one or more of the plurality of health data into windows of data.

12. The method of claim 11, wherein the plurality of first order features are determined by applying time differencing to two or more windows of data.

13. The method of any one of claim 1, wherein the plurality of first order features are determined by a smoothing function applied to at least a portion of the plurality of health data.

14. The method of claim 1, wherein the plurality of first order features are determined by applying a regression to at least a portion of the plurality of health data.

15. The method of claim 11, wherein the plurality of first order features comprises at least one of: an average, a minimum, a maximum, and a standard deviation applied to each window of data.

16. The method of claim 1, wherein the plurality of first order features comprises clinical determinations.

17. The method of claim 16, wherein the clinical determinations are made during a word recall assessment, the clinical determinations comprising at least one of immediate recall, delayed recall, time taken to recall each word, accuracy of words recalled, number of hesitations when recalling, errors while recalling, words recalled with and without cueing, voice volume, voice tone, voice pitch, dysarthria, speech disorder, and/or vocal tremor.

18. The method of claim 1, wherein the plurality of modalities comprises electroencephalography (EEG).

19. The method of claim 1, wherein the plurality of modalities comprises audio.

20. The method of claim 1, wherein the plurality of modalities comprises fMRI.

21. The method of claim 1, wherein the plurality of modalities comprises one or more drawing assessments.

22. The method of claim 1, wherein the plurality of modalities comprises an eye tracker.

23. The method of claim 1, wherein the plurality of modalities comprises a smart device.

24. The method of claim 1, wherein the plurality of modalities comprises an accelerometer.

25. The method of claim 1, wherein the plurality of modalities comprises a heartbeat sensor.

26. The method of claim 1, wherein the plurality of modalities comprises a galvanic response sensor.

27. The method of claim 1, wherein at least a portion of the plurality of health data and/or a portion of the plurality of first order features is received from an electronic health record (EHR).

28. The method of claim 4, wherein the synthesized at least one value comprises missing data from at least one of the plurality of modalities.

29. The method of claim 28, wherein the synthesized at least one value comprises one or more data points within one or more time series of data of the plurality of health data and/or the plurality of first order features.

30. The method of claim 4, wherein the synthesized at least one value comprises another modality not in the plurality of modalities.

31. The method of claim 30, wherein the synthesized at least one value comprises a synthesized fMRI image based on input from non-fMRI modalities.

32. The method of claim 30, wherein the synthesized at least one value comprises a synthesized electroencephalogram (EEG) signal based on input from non-EEG modalities.

33. The method of claim 1, wherein the one or more biomarker and/or health condition comprises two or more biomarkers and/or health conditions, the method further comprising:
determining, based on the two or more biomarkers and/or health conditions, one or more additional assessments for the patient, wherein results from the one or more additional assessments provide data to eliminate at least one biomarker and/or health condition as a potential diagnosis.

34. The method of claim 1, wherein the one or more biomarker and/or health condition is a brain health assessment.

35. The method of claim 1, wherein the plurality of health data comprises at least one of: time and space orientation questions, sentence completion questions, one or more depression and/or anxiety screen, a backward digit span test, a ball balancing assessment, dual tasking assessment, and/or delayed subjective recall.
